(19) United States
(12) Patent Application Publication
(10) Pub. No.: US 2025/0280867 A1
(43) Pub. Date: Sep. 11, 2025

(54) AEROSOL-GENERATING ARTICLE WITH THICK AND LIGHT WRAPPER

(71) Applicant: Philip Morris Products S.A., Neuchâtel (CH)

(72) Inventors: Enrico BINASSI, Lausanne (CH); Gianpaolo D'AMBRA, Castel Maggiore (IT); Valerio D'AMBROGI, Rome (IT); Enrico PIETROBUONI, Casalecchio di Reno (IT); Matteo BALBONI, Sala Bolognese (IT); Luca DINI, Modena (IT)

(73) Assignee: Philip Morris Products S.A., Neuchâtel (CH)

(21) Appl. No.: 18/861,720

(22) PCT Filed: Jul. 14, 2023
(86) PCT No.: PCT/EP2023/069631

§ 371 Date: Oct. 30, 2024

(30) Foreign Application Priority Data
Jul. 20, 2022 (EP) 22186027.3

(51) Int. Cl. A24D 1/02; A24D 1/20

(52) U.S. Cl. CPC A24D 1/02; A24D 1/20

(57) ABSTRACT

An aerosol-generating article is provided, including: an aerosol-forming substrate in an aerosol-forming substrate portion; and a substrate wrapper at least partly circumscribing the aerosol-forming substrate portion, the substrate wrapper including one or more layers having a same length in a longitudinal direction of the aerosol-generating article, the substrate wrapper having a thickness of 50 micrometers or more and a density of 450 kilograms per cubic meter or less, and the aerosol-forming substrate including at least 5 percent of aerosol former by weight on a dry weight basis. An aerosol-generating system including the aerosol-generating article and an aerosol-generating device is also provided.

The present disclosure relates to an aerosol-generating article. The present disclosure further relates to an aerosol-generating system.

It is known to provide an aerosol-generating device for generating an inhalable vapor. Such devices may heat an aerosol-forming substrate contained in an aerosol-generating article without burning the aerosol-forming substrate. The aerosol-generating article may have a rod shape for insertion of the aerosol-generating article into a heating chamber of the aerosol-generating device.

The aerosol-generating device may comprise a heating arrangement. The heating arrangement may be an induction heating arrangement and may comprise an induction coil configured to inductively heat a susceptor. The susceptor may be part of the device or may be part of the aerosol-generating article.

It would be desirable to provide a more efficient aerosol-generating article. It would be desirable to provide an aerosol-generating article which requires a smaller amount of aerosol-forming substrate. It would be desirable to provide a mechanically more robust aerosol-generating article. It would be desirable to provide an aerosol-generating article which can be used with existing aerosol-generating devices.

According to an embodiment of the invention there is provided an aerosol-generating article. The aerosol-generating article may comprise an aerosol-forming substrate in an aerosol-forming substrate portion. The aerosol-generating article may comprise a substrate wrapper at least partly circumscribing the aerosol-forming substrate portion. The substrate wrapper may comprise one or more layers having the same length in a longitudinal direction of the aerosol-generating article. The substrate wrapper may have a thickness of 50 micrometers or more. The substrate wrapper may have a density of 800 kilograms per cubic meter or less.

According to an embodiment of the invention there is provided an aerosol-generating article. The aerosol-generating article comprises an aerosol-forming substrate in an aerosol-forming substrate portion. The aerosol-generating article comprises a substrate wrapper at least partly circumscribing the aerosol-forming substrate portion. The substrate wrapper comprises one or more layers having the same length in a longitudinal direction of the aerosol-generating article. The substrate wrapper has a thickness of 50 micrometers or more. The substrate wrapper has a density of 800 kilograms per cubic meter or less.

By means of the thick and lightweight substrate wrapper having a thickness of 50 micrometers or more and a density of 800 kilograms per cubic meter or less, a peripheral portion at an outer circumference of the aerosol-forming substrate may be substituted by wrapper material of the thick wrapper while not changing the external diameter of the aerosol-generating article. The thicker wrapper may compensate for a thinner diameter of the aerosol-forming substrate portion such that the external diameter of the aerosol-generating article is not reduced. The most outer peripheral portion of the aerosol-forming substrate may not be satisfactorily heated when the aerosol-generating article is heated by an internal heating element located centrally in the aerosol-forming substrate portion, due to the distance to the heating element and limited thermal transfer. The non-satisfactorily heated portion of aerosol-forming substrate may not heat up sufficiently during use to participate in aerosolization. The thick wrapper may advantageously replace the non-satisfactorily heated portion of aerosol-forming substrate. An amount of aerosol-forming substrate may thus be saved. A more efficient aerosol-generating article may be provided.

The thick wrapper may reduce the diameter of the aerosol-forming substrate portion circumscribed by the thick wrapper. The reduced diameter of the aerosol-forming substrate portion may improve the thermal contact between the aerosol-forming substrate and a susceptor, other heating element, provided in the aerosol-forming substrate portion. A more efficient aerosol-generating article may be provided.

With the external diameter of the aerosol-generating article not being changed, an aerosol-generating article which can be used with existing aerosol-generating devices may be provided.

By using a thick and lightweight substrate wrapper, a thick wrapper which may still be conveniently processed on existing manufacturing lines may be provided. The thick and lightweight wrapper may be easily bendable due to being lightweight although being thick.

The thick and lightweight wrapper may be economically favorable due to requiring only little material for having a low density while being thick.

The thick and lightweight wrapper may be more rigid than a conventional thin wrapper. A mechanically more robust aerosol-generating article may be provided. A mechanically more robust aerosol-generating article may be particularly advantageous when the aerosol-generating article is arranged to be inserted into a heating chamber of an aerosol-generating device.

The combination of all of the one or more layers of the substrate wrapper may define an overall thickness of the substrate wrapper of 50 micrometers or more and an overall density of the substrate wrapper of 800 kilograms per cubic meter or less. At least one of the one or more layers of the substrate wrapper may have an individual thickness of 50 micrometers or more and an individual density of 800 kilograms per cubic meter or less. Each of the one or more layers of the substrate wrapper may have an individual thickness of 50 micrometers or more and an individual density of 800 kilograms per cubic meter or less.

The substrate wrapper may be arranged such that it does not extend beyond longitudinal ends of the aerosol-forming substrate portion in a direction parallel to the longitudinal direction of the aerosol-generating article.

The substrate wrapper may have a thickness of 60 micrometers or more, preferably 70 micrometers or more, more preferably 75 micrometers or more, more preferably 80 micrometers or more, more preferably 90 micrometers or more, more preferably 100 micrometers or more, more preferably 110 micrometers or more, more preferably 120 micrometers or more, more preferably 130 micrometers or more, more preferably 140 micrometers or more, more preferably 145 micrometers or more, more preferably 150 micrometers or more. The substrate wrapper may have a thickness of about 148 micrometers. The substrate wrapper may have a thickness of between 143 micrometers and 153 micrometers. The substrate wrapper may have a thickness of between 140 micrometers and 160 micrometers.

The substrate wrapper may have a uniform thickness that does not differ at any point by more than about 30 micrometers, or more than about 20 micrometers, or more than about 10 micrometers, or more than about 5 micrometers, or more than about 2 micrometers.

The ratio of substrate wrapper thickness to aerosol-forming substrate portion diameter may be in a range from about 1:120 to about 1:20, or about 1:100 to about 1:30, or about 1:80 to about 1:35, or about 1:60 to about 1:40.

The density of the substrate wrapper may be 750 kilograms per cubic meter or less, preferably 700 kilograms per cubic meter or less, more preferably 650 kilograms per cubic meter or less, more preferably 600 kilograms per cubic meter or less, more preferably 550 kilograms per cubic meter or less, more preferably 500 kilograms per cubic meter or less, more preferably 450 kilograms per cubic meter or less. The density of the substrate wrapper may be 400 kilograms per cubic meter or less, preferably 350 kilograms per cubic meter or less, more preferably about 320 kilograms per cubic meter.

The density of the substrate wrapper may be 400 kilograms per cubic meter or less and the substrate wrapper may have a thickness of 60 micrometers or more, preferably 70 micrometers or more, more preferably 75 micrometers or more, more preferably 80 micrometers or more, more preferably 90 micrometers or more, more preferably 100 micrometers or more, more preferably 110 micrometers or more, more preferably 120 micrometers or more, more preferably 130 micrometers or more, more preferably 140 micrometers or more, more preferably 145 micrometers or more, more preferably 150 micrometers or more. The substrate wrapper may have a thickness of about 148 micrometers. The density of the substrate wrapper may be 400 kilograms per cubic meter or less and the substrate wrapper may have a thickness of between 143 micrometers and 153 micrometers. The density of the substrate wrapper may be 400 kilograms per cubic meter or less and the substrate wrapper may have a thickness of between 140 micrometers and 160 micrometers.

The basis weight of the substrate wrapper may be less than 60 grams per square meter. The basis weight of the substrate wrapper may be more than 28 grams per square meter and less than 60 grams per square meter. The basis weight of the substrate wrapper may be more than 45 grams per square meter and less than 60 grams per square meter.

The basis weight of the substrate wrapper may be less than 50 grams per square meter. The basis weight of the substrate wrapper may be more than 28 grams per square meter and less than 50 grams per square meter. The basis weight of the substrate wrapper may be more than 45 grams per square meter and less than 50 grams per square meter. The basis weight of the substrate wrapper may be about 48 grams per square meter.

The substrate wrapper may have a thickness of more than 145 micrometers and a density of 400 kilograms per cubic meter or less.

The substrate wrapper may comprise one or more perforations or may not comprise any perforations.

The substrate wrapper may exhibit a permeability of the wrapper of more than 10 CORESTA units, more than 20 CORESTA units, more than 50 CORESTA units, more than 100 CORESTA units, more than 500 CORESTA units, more than 1000 CORESTA units, more than 1500 CORESTA units, more than 2000 CORESTA units, more than 2500 CORESTA units, more than 3000 CORESTA units, more than 3500 CORESTA units, or more than 4000 CORESTA units. The substrate wrapper may exhibit a permeability of the wrapper of between 10 CORESTA units and 10,000 CORESTA units, preferably between 50 CORESTA units and 8000 CORESTA units, more preferably between 100 CORESTA units and 5000 CORESTA units. The substrate wrapper may exhibit a permeability of the wrapper of between 4000 CORESTA units and 4800 CORESTA units, preferably between 4200 CORESTA units and 4600 CORESTA units, more preferably between 4300 CORESTA units and 4500 CORESTA units.

The substrate wrapper may have a thickness of more than 145 micrometers, a density of 400 kilograms per cubic meter or less, and a permeability of the wrapper of between 50 CORESTA units and 5000 CORESTA units, preferably between 4200 CORESTA units and 4600 CORESTA units, more preferably between 4300 CORESTA units and 4500 CORESTA units.

The permeability of the substrate wrapper may be determined by utilizing the International Standard test method ISO 2965:2009 and the result may be presented as cubic centimeters per minute per square centimeters and referred to as “CORESTA units”.

The aerosol generating-article may comprise an additional wrapper circumscribing the substrate wrapper. The additional wrapper may exhibit a permeability of the wrapper of less than 100 CORESTA units, less than 80 CORESTA units, less than 50 CORESTA units, less than 40 CORESTA units, or less than 30 CORESTA units. The permeability of the additional wrapper may be less than the permeability of the substrate wrapper. The permeability of the additional wrapper may be less than 1%, less than 2%, less than 5%, less than 10%, or less than 20% of the permeability of the substrate wrapper. The permeability of the additional wrapper may be less than 50 CORESTA units and the permeability of the substrate wrapper may be between 4000 CORESTA units and 4800 CORESTA units, preferably between 4200 CORESTA units and 4600 CORESTA units, more preferably between 4300 CORESTA units and 4500 CORESTA units. The additional wrapper may be a tipping wrapper as described herein. The additional wrapper may be a combining wrapper. The additional wrapper may advantageously reduce the overall permeability where a substrate wrapper with a high permeability is used.

The substrate wrapper may be embossed or may not be embossed. The substrate wrapper may be both perforated and embossed.

The term “embossment” is used herein to refer to protrusions formed in the surface of a wrapper. These protrusions may be carved, moulded or stamped into the wrapper. The portion of wrapper carrying such embossments is said to be embossed.

The substrate wrapper may comprise an embossed portion. The embossed portion of the substrate wrapper may have one embossment. The embossed portion of the substrate wrapper may have a plurality of embossments. The one or more embossments may have a depth of from 0.07 millimeter to 0.21 millimeter, preferably from 0.10 millimeter to 0.18 millimeter and more preferably from 0.12 millimeter to 0.16 millimeter. Each embossment may also have a pitch of from 0.2 millimeter to 0.4 millimeter, preferably from 0.25 millimeter to 0.35 millimeter, more preferably from 0.275 millimeter to 0.325 millimeter.

The substrate wrapper may have a roughness of between about 50 Bekk seconds and about 1000 Bekk seconds, preferably between about 100 Bekk seconds and about 200 Bekk seconds. The roughness expressed in Bekk seconds may be measured by means of a standard test using a BEKK Smoothness Tester, which creates a vacuum and measures the time it takes for the vacuum to drop from 50.66 kPa to 48.00 kPa. The test is recognized by the international standard ISO 5627.

As used herein, ‘the total density of the aerosol-forming substrate portion’ refers to the total mass of material received within the volume which is circumscribed by the substrate wrapper, divided by the volume which is circumscribed by the substrate wrapper. The mass of the substrate wrapper itself and optional further wrappers circumscribing the substrate wrapper is not taken into account. The volume of the substrate wrapper itself and optional further wrappers circumscribing the substrate wrapper is not taken into account.

The total density of the aerosol-forming substrate portion may be determined after conditioning the aerosol-generating article in accordance to ISO Standard 3402:1999. The aerosol-forming substrate is removed from the aerosol-forming substrate portion and is weighed. If a susceptor is present, the susceptor is also removed from the aerosol-forming substrate portion and is also weighed. The inner volume of the aerosol-forming substrate portion is determined. This may be done, for example, by laser measurement. The inner volume of the aerosol-forming substrate portion generally corresponds to the cylindrical volume within the substrate wrapper. The total density of the aerosol-forming substrate portion is calculated by dividing the sum of the mass of the aerosol-forming substrate and, if present, the mass of the susceptor by the inner volume of the substrate portion. This may be repeated 20 times for 20 different individual aerosol-generating articles to receive an average value.

As used herein, ‘the total density of the aerosol-generating article at the longitudinal position of the aerosol-forming substrate portion’ refers to the total mass of material received within the volume which is defined by the mean transversal cross-sectional area of the aerosol-generating article along the length of the aerosol-forming substrate portion, divided by the said volume. The mass of each of the aerosol-forming substrate, an optional susceptor, the substrate wrapper, and each one or more optional further wrappers circumscribing the substrate wrapper is taken into account. The volume of the substrate wrapper itself and each of the one or more optional further wrappers circumscribing the substrate wrapper is taken into account.

The total density of the aerosol-generating article at the longitudinal position of the aerosol-forming substrate portion' may be determined after conditioning the aerosol-generating article in accordance to ISO Standard 3402:1999.

The thickness of the substrate wrapper may be determined in accordance to ISO 534:2011. The density of the substrate wrapper may be determined in accordance to ISO 534:2011. The thickness of the substrate wrapper may be determined in accordance to ASTM E252-06(2021)e1. Generally, for embossed substrate wrappers, the local thickness at a position of an embossment may be less than the thickness at a position without an embossment. As used herein, for embossed wrappers, the thickness of the substrate wrapper refers to the thickness at positions without embossments. For embossed wrappers, the thickness of the substrate wrapper may be determined before the wrapper is being embossed.

Unless defined otherwise, all measurements described herein are performed after conditioning the samples in accordance to ISO Standard 3402:1999.

The density of the substrate wrapper may be calculated by dividing the basis weight of the substrate wrapper by the thickness of the substrate wrapper. The basis weight, also called grammage, refers to the mass of the substrate wrapper per sheet size, usually expressed in grams per square meter. The basis weight may be obtained, for example, by weighing a 1 square meter sized sheet of the substrate wrapper.

As used herein, when referring to the substrate wrapper, the term “lightweight” means that the density of the substrate wrapper is 800 kilograms per cubic meter or less, preferably 750 kilograms per cubic meter or less, more preferably 700 kilograms per cubic meter or less, more preferably 650 kilograms per cubic meter or less, more preferably 600 kilograms per cubic meter or less, more preferably 550 kilograms per cubic meter or less, more preferably 500 kilograms per cubic meter or less, more preferably 450 kilograms per cubic meter or less, more preferably 400 kilograms per cubic meter or less, more preferably 350 kilograms per cubic meter or less, more preferably is about 320 kilograms per cubic meter.

As used herein, when referring to the substrate wrapper, the term “thick” means that a thickness of the substrate wrapper is 50 micrometers or more, preferably 60 micrometers or more, more preferably 70 micrometers or more, more preferably 75 micrometers or more, more preferably 80 micrometers or more, more preferably 90 micrometers or more, more preferably 100 micrometers or more, more preferably 110 micrometers or more, more preferably 120 micrometers or more, more preferably 130 micrometers or more, more preferably 140 micrometers or more, more preferably 145 micrometers or more, more preferably 150 micrometers or more.

The substrate wrapper may extend along the entire length of the aerosol-forming substrate portion in a direction along the longitudinal axis of the aerosol-generating article. The substrate wrapper may extend along at least 40 percent, preferably at least 50 percent, more preferably at least 60 percent, more preferably at least 70 percent, more preferably at least 80 percent, more preferably at least 90 percent, more preferably at least 95 percent of the length of the aerosol-forming substrate portion in a direction along the longitudinal axis of the aerosol-generating article.

The substrate wrapper may be in direct physical contact with the aerosol-forming substrate. In that case, there is no layer of material between the substrate wrapper and the aerosol-forming substrate.

The substrate wrapper may be formed from a single continuous sheet of material. The single continuous sheet may be wrapped around the aerosol-forming substrate portion by about one turn. Generally, the single continuous sheet may be wrapped around the substrate portion by slightly more than one turn in order to form an overlapping region of opposing end portions of the substrate wrapper. The thickness of the wrapper is not to be measured in the overlapping region. The substrate wrapper formed from a single continuous sheet of material may thus comprise only a single layer, except for an optional overlapping region, if present.

The substrate wrapper may be formed from a single continuous sheet which is wrapped around the aerosol-forming substrate portion by at least about two or more turns. In that case, two or more layers of substrate wrapper are wrapped around the aerosol-forming substrate portion—not taking into account an additional overlapping region formed by the overlapping opposing end portions of the wrapper. In that case, the thickness of the substrate wrapper may be obtained by multiplying the thickness of an individual layer, i.e. the sheet thickness, by the number of turns. The thickness of the substrate wrapper is not to be obtained by multiplying the thickness of an individual layer by the number of turns in the overlapping region formed by overlapping opposing end portions of the wrapper. None of the individual layers extends beyond ends of the aerosol-forming substrate portion in a longitudinal direction of the aerosol-generating article.

The substrate wrapper may comprise one or more of cardboard, plastics, and metal foil.

The substrate wrapper may comprise a cellulosic material, for example one or more of paper, wood, textile, natural fibers, and artificial fibers. The substrate wrapper may comprise a paper layer. The substrate wrapper may be made of a single paper sheet. The substrate wrapper may comprise a single paper layer wrapped around the aerosol-forming substrate portion, except for an option overlapping portion. The substrate wrapper may be made of a single paper sheet wrapped around the aerosol-forming substrate portion two or more times, resulting in a substrate wrapper comprising two or more layers having the same length.

The substrate wrapper may be a paper wrapper or a non-paper wrapper. Suitable non-paper wrappers include, but are not limited to sheets of homogenised tobacco materials.

The substrate wrapper may comprise a laminate sheet. The substrate wrapper may be made of a single laminate sheet. The laminate sheet may be a laminate of a paper layer with an aluminum layer.

The wrapper may be formed of a laminate material comprising a plurality of layers. The wrapper may be formed of a metallic co-laminated sheet, for example an aluminium co-laminated sheet. The metallic layer of the co-laminated sheet may have a grammage from 12 grams per square meter to 25 grams per square meter, preferably from 15 grams per square meter to 20 grams per square meter. The metallic layer of the co-laminated sheet may have a thickness from 2 micrometers to 15 micrometers, preferably from 3 micrometers to 12 micrometers, more preferably from 5 micrometers to 10 micrometers.

The substrate wrapper may be a paper wrapper comprising PVOH (polyvinyl alcohol) or silicone (or polysiloxane) (or polysiloxane). Addition of PVOH (polyvinyl alcohol) or silicone (or polysiloxane) may improve the grease barrier properties of the wrapper.

The substrate wrapper may comprise a flame retardant composition comprising one or more flame retardant compounds. The term “flame retardant compounds” is used herein to describe chemical compounds that, when added to or otherwise incorporated into a carrier substrate, such as paper or plastic compounds, provide the carrier substrate with varying degrees of flammability protection.

A number of suitable flame retardant compounds are known to the skilled person. In particular, several flame retardant compounds and formulations suitable for treating cellulosic materials are known and have been disclosed and may find use in the manufacture of wrappers for aerosol-generating articles in accordance with the present invention.

The substrate wrapper may be a substrate wrapper system formed from two or more individual substrate wrapper sub-sheets. In that case, the thickness of the substrate wrapper may be obtained by adding the thicknesses of the individual substrate wrapper sub-sheets of the substrate wrapper system. It may be that none of the individual substrate wrapper sub-sheets extends beyond ends of the aerosol-forming substrate portion in a longitudinal direction of the aerosol-generating article. Each of the individual substrate wrapper sub-sheets forming the substrate wrapper system may be of the same length in a direction parallel to the longitudinal axis of the aerosol-generating article.

The substrate wrapper may be a substrate wrapper system formed from two or more individual substrate wrapper sub-sheets, wherein each of the two or more individual substrate wrapper sub-sheets at least partly circumscribes the aerosol-forming substrate portion, wherein none of the two or more individual substrate wrapper sub-sheets extends beyond ends of the aerosol-forming substrate portion in a longitudinal direction of the aerosol-generating article, wherein each of the two or more individual substrate wrapper sub-sheets has a thickness of 50 micrometers or more and a density of 800 kilograms per cubic meter or less, and, preferably, wherein each of the individual substrate wrapper sub-sheets forming the substrate wrapper system is of the same length in a direction parallel to the longitudinal axis of the aerosol-generating article.

The substrate wrapper may be a substrate wrapper system formed from two or more individual substrate wrapper sub-sheets, wherein each of the two or more individual substrate wrapper sub-sheets at least partly circumscribes the aerosol-forming substrate portion, wherein each of the two or more individual substrate wrapper sub-sheets has the same length in a longitudinal direction of the aerosol-generating article, wherein each of the two or more individual substrate wrapper sub-sheets has a thickness of 50 micrometers or more and a density of 800 kilograms per cubic meter or less.

The substrate wrapper may be a substrate wrapper system formed from two or more individual substrate wrapper sub-sheets, wherein each of the two or more individual substrate wrapper sub-sheets at least partly circumscribes the aerosol-forming substrate portion, wherein none of the two or more individual substrate wrapper sub-sheets extends beyond ends of the aerosol-forming substrate portion in a longitudinal direction of the aerosol-generating article, wherein the sum of the two or more individual substrate wrapper sub-sheets has a thickness of 50 micrometers or more and a density of 800 kilograms per cubic meter or less, and, preferably, wherein each of the individual substrate wrapper sub-sheets forming the substrate wrapper system is of the same length in a direction parallel to the longitudinal axis of the aerosol-generating article.

The substrate wrapper may be a substrate wrapper system formed from two or more individual substrate wrapper sub-sheets, wherein each of the two or more individual substrate wrapper sub-sheets at least partly circumscribes the aerosol-forming substrate portion, wherein each of the two or more individual substrate wrapper sub-sheets has the same length in a longitudinal direction of the aerosol-generating article, wherein the sum of the two or more individual substrate wrapper sub-sheets has a thickness of 50 micrometers or more and a density of 800 kilograms per cubic meter or less.

The substrate wrapper system may be formed from two individual sheets. The substrate wrapper system may be formed from a first individual sheet and a second individual sheet. The first individual sheet may be provided by a first wrapper comprising a first overlapping region formed by overlapping opposing end portions of the first wrapper. The second individual sheet may be provided by a second wrapper comprising a second overlapping region formed by overlapping opposing end portions of the second wrapper. The first overlapping region may be offset from the second overlapping region by at least about 5 percent of a circumference of the aerosol-forming substrate portion, preferably by at least about 10 percent of a circumference of the aerosol-forming substrate portion, more preferably by at least about 15 percent of a circumference of the aerosol-forming substrate portion, more preferably by about 40 percent to about 60 percent of a circumference of the aerosol-forming substrate portion. One or both of the first and second individual sheets may be paper wrappers.

The aerosol-generating article may comprise a downstream section located downstream of the aerosol-forming substrate portion. The downstream section is preferably located immediately downstream of the aerosol-forming substrate portion. The downstream section of the aerosol-generating article preferably extends between the aerosol-forming substrate portion and the downstream end of the aerosol-generating article. The downstream section may comprise one or more elements, each of which will be described in more detail within the present disclosure.

A length of the downstream section may be at least 10 millimeters, or at least 20 millimeters, or at least 25 millimeters, or at least 30 millimeters.

A length of the downstream section may be less than 70 millimeters, or less than 60 millimeters, or less than 50 millimeters.

For example, a length of the downstream section may be between 20 millimeters and 70 millimeters, or between 25 millimeters and 60 millimeters, or between 30 millimeters and 50 millimeters.

The downstream section of an aerosol-generating article according to the present invention preferably comprises a hollow tubular cooling element provided downstream of the aerosol-forming substrate portion. The hollow tubular cooling element may advantageously provide an aerosol-cooling element for the aerosol-generating article. The hollow tubular cooling element may be provided immediately downstream of the aerosol-forming substrate portion. In other words, the hollow tubular cooling element may abut a downstream end of the aerosol-forming substrate portion. The hollow tubular cooling element may define an upstream end of the downstream section of the aerosol-generating article. The downstream end of the aerosol-generating article may coincide with the downstream end of the downstream section. In some embodiments, the downstream section of the aerosol-generating article comprises a single hollow tubular element. In other words, the downstream section of the aerosol-generating article may comprise only one hollow tubular element. In other embodiments, the downstream section comprises two or more hollow tubular elements, as described below.

As used throughout the present disclosure, the term “hollow tubular element” denotes a generally elongate element defining a lumen or airflow passage along a longitudinal axis thereof. In particular, the term “tubular” will be used in the following with reference to a tubular element having a substantially cylindrical cross-section and defining at least one airflow conduit establishing an uninterrupted fluid communication between an upstream end of the tubular element and a downstream end of the tubular element. However, it will be understood that alternative geometries (for example, alternative cross-sectional shapes) of the tubular element may be possible. The hollow tubular cooling element may be an individual, discrete element of the aerosol-generating article which has a defined length and thickness.

In the context of the present invention, a hollow tubular cooling element provides an unrestricted flow channel. This means that the hollow tubular cooling element provides a negligible level of resistance to draw (RTD). The term “negligible level of RTD” is used to describe an RTD of less than 1 millimeters of water gauge per 10 millimeters of length of the hollow tubular cooling element, preferably less than 0.4 millimeters of water gauge per 10 millimeters of length of the hollow tubular cooling element, more preferably less than 0.1 millimeters of water gauge per 10 millimeters of length of the hollow tubular cooling element.

The RTD of a hollow tubular cooling element is preferably less than or equal to 10 millimeters of water gauge, or less than or equal to 5 millimeters of water gauge, or less than or equal to 2.5 millimeters of water gauge, or less than or equal to 2 millimeters of water gauge, or less than or equal to 1 millimeter of water gauge.

The RTD of a hollow tubular cooling element may be at least 0 millimeters of water gauge, or at least 0.25 millimeters of water gauge or at least 0.5 millimeters of water gauge or at least 1 millimeter of water gauge.

In aerosol-generating articles in accordance with the present invention the overall RTD of the article depends essentially on the RTD of the rod and optionally on the RTD of the downstream and/or upstream elements. This is because the hollow tubular cooling element is substantially empty and, as such, substantially only marginally contribute to the overall RTD of the aerosol-generating article.

The flow channel should therefore be free from any components that would obstruct the flow of air in a longitudinal direction. Preferably, the flow channel is substantially empty and particularly preferably the flow channel is empty.

As will be described in greater detail within the present disclosure, the aerosol-generating article may comprise a ventilation zone at a location along the downstream section. In some embodiments, the aerosol-generating article may comprise a ventilation zone at a location along the hollow tubular cooling element. Such, or any, ventilation zone may extend through the peripheral wall of the hollow tubular cooling element. As such, fluid communication is established between the flow channel internally defined by the hollow tubular cooling element and the outer environment. The ventilation zone is further described within the present disclosure.

The length of the hollow tubular cooling element may be at least 15 millimeters, or at least 20 millimeters, or at least 25 millimeters. The length of the hollow tubular cooling element may be less than 50 millimeters, or less than 45 millimeters, or less than 40 millimeters. For example, the length of the hollow tubular cooling element may be between 15 millimeters and 50 millimeters, or between 20 millimeters and 45 millimeters, or between 20 millimeters and 40 millimeters, or between 20 millimeters and 30 millimeters, or between 25 millimeters and 40 millimeters.

A relatively long hollow tubular cooling element provides and defines a relatively long internal cavity within the aerosol-generating article and downstream of the aerosol-forming substrate portion. Providing an empty cavity downstream (preferably, immediately downstream) of the aerosol-forming substrate enhances the nucleation of aerosol particles generated by the substrate. Providing a relatively long cavity maximises such nucleation benefits, thereby improving aerosol formation and cooling.

The wall thickness of the hollow tubular cooling element may between 100 micrometers and 2 millimeters, or between 150 micrometers and 1.5 millimeters, or between 200 micrometers and 1.25 millimeters.

The hollow tubular cooling element preferably has an external diameter that is approximately equal to the external diameter of the aerosol-generating article.

The hollow tubular cooling element may have an external diameter of between 5 millimeters and 10 millimeters, for example of between 5.5 millimeters and 9 millimeters or of between 6 millimeters and 8 millimeters. In certain embodiments, the hollow tubular cooling element has an external diameter of less than 7 millimeters.

The hollow tubular cooling element may have an internal diameter. Preferably, the hollow tubular cooling element has a constant internal diameter along a length of the hollow tubular cooling element. However, the internal diameter of the hollow tubular cooling element may vary along the length of the hollow tubular cooling element.

The hollow tubular cooling element may have an internal diameter of at least 2 millimeters. For example, the hollow tubular cooling element may have an internal diameter of at least 3 millimeters, at least 4 millimeters, or at least 5 millimeters.

The provision of a hollow tubular cooling element having an internal diameter as set out above may advantageously provide sufficient rigidity and strength to the hollow tubular cooling element.

The hollow tubular cooling element may have an internal diameter of no more than 10 millimeters. For example, the hollow tubular cooling element may have an internal diameter of no more than 9 millimeters, no more than 8 millimeters, or no more than 7 millimeters.

The provision of a hollow tubular cooling element having an internal diameter as set out above may advantageously reduce the resistance to draw of the hollow tubular cooling element.

The hollow tubular cooling element may have an internal diameter of between 2 millimeters and 10 millimeters, between 3 millimeters and 9 millimeters, between 4 millimeters and 8 millimeters, or between 5 millimeters and 7 millimeters.

The lumen or cavity of the hollow tubular cooling element may have any cross sectional shape. The lumen of the hollow tubular cooling element may have a circular cross sectional shape.

The hollow tubular cooling element may comprise a paper-based material. The hollow tubular cooling element may comprise at least one layer of paper. The paper may be very rigid paper. The paper may be crimped paper, such as crimped heat resistant paper or crimped parchment paper.

Preferably, the hollow tubular cooling element may comprise cardboard. The hollow tubular cooling element may be a cardboard tube. The hollow tubular cooling element may be formed from cardboard. Advantageously, cardboard is a cost-effective material that provides a balance between being deformable in order to provide ease of insertion of the article into an aerosol-generating device and being sufficiently stiff to provide suitable engagement of the article with the interior of the device. A cardboard tube may therefore provide suitable resistance to deformation or compression during use.

The hollow tubular cooling element may be a paper tube. The hollow tubular cooling element may be a tube formed from spirally wound paper. The hollow tubular cooling element may be formed from a plurality of layers of the paper. The paper may have a basis weight of at least 50 grams per square meter, at least 60 grams per square meter, at least 70 grams per square meter, or at least 90 grams per square meter.

The hollow tubular cooling element may comprise a polymeric material. For example, the hollow tubular cooling element may comprise a polymeric film. The polymeric film may comprise a cellulosic film. The hollow tubular cooling element may comprise low density polyethylene (LDPE) or polyhydroxyalkanoate (PHA) fibres. The hollow tube may comprise cellulose acetate tow.

Where the hollow tubular cooling element comprises cellulose acetate tow, the cellulose acetate tow may have a denier per filament of between 2 and 4 and a total denier of between 25 and 40.

Preferably, the aerosol-generating article according to the present invention comprises a ventilation zone at a location along the downstream section. In more detail, in those embodiments wherein the downstream section comprises a hollow tubular cooling element, the ventilation zone may be provided at a location along the hollow tubular cooling element. Alternatively or in addition, in those embodiments where the downstream section comprises a downstream hollow tubular element, as described below, the ventilation zone may be provided at a location along the downstream hollow tubular element.

As such, a ventilated cavity is provided downstream of the aerosol-forming substrate portion. This provides several potential technical benefits. First of all, the inventors have found that one such ventilated hollow tubular cooling element provides a particularly efficient cooling of the aerosol. Secondly, the inventors have surprisingly found that such rapid cooling of the volatile species released upon heating the aerosol-forming substrate enhances nucleation of aerosol particles.

The ventilation zone may typically comprise a plurality of perforations through the peripheral wall of the hollow tubular cooling element. Preferably, the ventilation zone comprises at least one circumferential row of perforations. In some embodiments, the ventilation zone may comprise two circumferential rows of perforations. For example, the perforations may be formed online during manufacturing of the aerosol-generating article. Preferably, each circumferential row of perforations comprises from 8 to 30 perforations.

An aerosol-generating article in accordance with the present invention may have a ventilation level of at least 40 percent. Increasing the ventilation level may increase the level of aerosol cooling. However, increasing the ventilation level may mean that less air is admitted into the aerosol-generating article via the upstream end of the aerosol-generating article which then flows through the aerosol-forming substrate portion. The ventilation level may thereby be selected based on a desired temperature and composition of the aerosol delivered to a user.

The aerosol-generating article preferably has a ventilation level of at least 45 percent, more preferably at least 50 percent, more preferably at least 60 percent, more preferably at least 70 percent.

An aerosol-generating article in accordance with the present invention may have a ventilation level of less than or equal to 90 percent, more preferably less than or equal to 85 percent, more preferably less than or equal to 80 percent.

Thus, an aerosol-generating article in accordance with the present invention may have a ventilation level from 45 percent to 90 percent, more preferably from 45 percent to 85 percent, even more preferably from 45 percent to 80 percent. The aerosol-generating article in accordance with the present invention may have a ventilation level from 50 percent to 90 percent, preferably from 50 percent to 85 percent, more preferably from 50 percent to 80 percent. The aerosol-generating article in accordance with the present invention may have a ventilation level from 60 percent to 90 percent, preferably from 60 percent to 85 percent, more preferably from 60 percent to 80 percent. The aerosol-generating article in accordance with the present invention may have a ventilation level from 70 percent to 90 percent, preferably from 70 percent to 85 percent, more preferably from 70 percent to 80 percent.

For example, the aerosol-generating article may have a ventilation level of about 75 percent.

As discussed in the present disclosure, the downstream section may comprise a downstream filter segment. The downstream filter segment may extend to a downstream end of the downstream section. The downstream filter segment may be located at the downstream end of the aerosol-generating article. The downstream end of the downstream filter segment may define the downstream end of the aerosol-generating article. The downstream filter segment may also be referred to as mouth-end filter.

The downstream filter segment may be located downstream of a hollow tubular cooling element, which is described above. The downstream filter segment may extend between the hollow tubular cooling element and the downstream end of the aerosol-generating article.

The downstream filter segment is preferably a solid plug, which may also be described as a ‘plain’ plug and is non-tubular. The filter segment therefore preferably has a substantially uniform transverse cross section.

The downstream filter segment is preferably formed of a fibrous filtration material. The fibrous filtration material may be for filtering the aerosol that is generated from the aerosol-forming substrate. Suitable fibrous filtration materials would be known to the skilled person. Particularly preferably, the at least one downstream filter segment comprises a cellulose acetate filter segment formed of cellulose acetate tow.

In certain preferred embodiments, the downstream section includes a single downstream filter segment. In alternative embodiments, the downstream section includes two or more downstream filter segments axially aligned in an abutting end to end relationship with each other.

The downstream filter segment may optionally comprise a flavourant, which may be provided in any suitable form. For example, the downstream filter segment may comprise one or more capsules, beads or granules of a flavourant, or one or more flavour loaded threads or filaments.

Preferably, the downstream filter segment has a low particulate filtration efficiency.

Preferably, the downstream filter segment is circumscribed by a plug wrap. Preferably, the downstream filter segment is unventilated such that air does not enter the aerosol-generating article along the downstream filter segment.

The downstream filter segment is preferably connected to one or more of the adjacent upstream components of the aerosol-generating article by means of a tipping wrapper.

The downstream filter segment preferably has an external diameter that is approximately equal to the external diameter of the aerosol-generating article. The external diameter of a downstream filter segment may be substantially the same as the external diameter of the hollow tubular cooling element.

The external diameter of the downstream filter segment may be between 5 millimeters and 10 millimeters, or between 5.5 millimeters and 9 millimeters, or between 6 millimeters and 8 millimeters. In certain embodiments, the external of the downstream filter segment is less than 7 millimeters.

Unless otherwise specified, the resistance to draw (RTD) of a component or the aerosol-generating article is measured in accordance with ISO 6565-2015. The RTD refers the pressure required to force air through the full length of a component. The terms “pressure drop” or “draw resistance” of a component or article may also refer to the “resistance to draw”. Such terms generally refer to the measurements in accordance with ISO 6565-2015 normally carried out at a volumetric flow rate of 17.5 milliliters per second at the output or downstream end of the measured component at a temperature of 22 degrees Celsius, a pressure of 101 kPa (about 760 Torr) and a relative humidity of 60%. Conditions for smoking and smoking machine specifications are set out in ISO Standard 3308 (ISO 3308:2000). Atmosphere for conditioning and testing are set out in ISO Standard 3402 (ISO 3402:1999).

The resistance to draw (RTD) may be expressed with the units of pressure “millimeter(s) of water gauge” (mmWG).

The resistance to draw (RTD) of the downstream section may be at least 0 millimeters of water gauge. The RTD of the downstream section may be at least 3 millimeters of water gauge. The RTD of the downstream section may be at least 6 millimeters of water gauge. The RTD of the downstream section may be no greater than 12 millimeters of water gauge. The RTD of the downstream section may be no greater than 11 millimeters of water gauge. The RTD of the downstream section may be no greater than 10 millimeters of water gauge.

The resistance to draw (RTD) characteristics of the downstream section may be wholly or mostly attributed to the RTD characteristics of the downstream filter segment of the downstream section. In other words, the RTD of the downstream filter segment of the downstream section may wholly define the RTD of the downstream section.

The resistance to draw (RTD) of the downstream filter segment may be at least 0 millimeters of water gauge, or at least 3 millimeters of water gauge, or at least 6 millimeters of water gauge. The RTD of the downstream filter segment may be no greater than 12 millimeters of water gauge, or no greater than 11 millimeters of water gauge, or no greater than 10 millimeters of water gauge.

As mentioned above, the downstream filter segment may be formed of a fibrous filtration material. The downstream filter segment may be formed of a porous material. The downstream filter segment may be formed of a biodegradable material. The downstream filter segment may be formed of a cellulose material, such as cellulose acetate. For example, a downstream filter segment may be formed from a bundle of cellulose acetate fibres having a denier per filament between 10 and 15. For example, a downstream filter segment formed from relatively low density cellulose acetate tow, such as cellulose acetate tow comprising fibres of 12 denier per filament.

The downstream filter segment may be formed of a polylactic acid based material. The downstream filter segment may be formed of a bioplastic material, preferably a starch-based bioplastic material. The downstream filter segment may be made by injection moulding or by extrusion. Bioplastic-based materials are advantageous because they are able to provide downstream filter segment structures which are simple and cheap to manufacture with a particular and complex cross-sectional profile, which may comprise a plurality of relatively large air flow channels extending through the downstream filter segment material, that provides suitable RTD characteristics.

The length of the downstream filter segment may be at least 5 millimeters, or at least 10 millimeters. The length of the downstream filter segment may be less than 25 millimeters, or less than 20 millimeters. For example, the length of the downstream filter segment may be between 5 millimeters and 25 millimeters, or between 10 millimeters and 25 millimeters, or between 5 millimeters and 20 millimeters, or between 10 millimeters and 20 millimeters.

The downstream section may further comprise one or more additional hollow tubular elements.

In certain embodiments, the downstream section may comprise a hollow tubular support element upstream of the hollow tubular cooling element described above. Preferably, the hollow tubular support element abuts the downstream end of the aerosol-forming substrate portion. Preferably, the hollow tubular support element abuts the upstream end of the hollow tubular cooling element. Preferably, the hollow tubular support element and the hollow tubular cooling element are adjacent to each other and together provide a hollow tubular section within the downstream section.

The hollow tubular support element may be formed from any suitable material or combination of materials. For example, the support element may be formed from one or more materials selected from the group consisting of: cellulose acetate; cardboard; crimped paper, such as crimped heat resistant paper or crimped parchment paper; and polymeric materials, such as low density polyethylene (LDPE). In a preferred embodiment, the support element is formed from cellulose acetate. Other suitable materials include polyhydroxyalkanoate (PHA) fibres. In a preferred embodiment, the hollow tubular support element comprises a hollow acetate tube.

The hollow tubular support element preferably has an external diameter that is approximately equal to the external diameter of the aerosol-generating article.

The hollow tubular support element may have an external diameter of between 5 millimeters and 10 millimeters, for example of between 5.5 millimeters and 9 millimeters or of between 6 millimeters and 8 millimeters. In a preferred embodiment, the hollow tubular support element has an external diameter of less than 7 millimeters.

The hollow tubular support element may have a wall thickness of at least 1 millimeter, preferably at least 1.5 millimeters, more preferably at least 2 millimeters.

The hollow tubular support element may have a length of at least 5 millimeters. Preferably, the support element has a length of at least 6 millimeters, more preferably at least 7 millimeters.

The hollow tubular support element may have a length of less than 15 millimeters. Preferably, the hollow tubular support element has a length of less than 12 millimeters, more preferably less than 10 millimeters.

In some embodiments, the hollow tubular support element has a length from 5 millimeters to 15 millimeters, preferably from 6 millimeters to 15 millimeters, more preferably from 7 millimeters to 15 millimeters. In other embodiments, the hollow tubular support element has a length from 5 millimeters to 12 millimeters, preferably from 6 millimeters to 12 millimeters, more preferably from 7 millimeters to 12 millimeters. In further embodiments, the support element has a length from 5 millimeters to 10 millimeters, preferably from 6 millimeters to 10 millimeters, more preferably from 7 millimeters to 10 millimeters.

Alternatively or in addition to the hollow tubular support element, the downstream section may further comprise a downstream hollow tubular element downstream of the hollow tubular cooling element. The downstream hollow tubular element may be provided immediately adjacent to the hollow tubular cooling element. Alternatively and preferably, the downstream hollow tubular element is separated from the hollow tubular cooling element by at least one other component. For example, the downstream section may comprise a downstream filter segment between the hollow tubular cooling element and the downstream hollow tubular element.

The downstream hollow tubular element preferably extends to the downstream end of the downstream section. The downstream hollow tubular element therefore preferably extends to the downstream end of the aerosol-generating article. Where the downstream hollow tubular element extends to the downstream end of the aerosol-generating article, the downstream hollow tubular element may define a mouth end cavity of the aerosol-generating article.

In certain embodiments, an additional downstream hollow tubular element may be provided, so that the downstream section comprises two adjacent downstream hollow tubular elements, downstream of the downstream filter segment.

The RTD of the downstream hollow tubular element may be less than or equal to 10 millimeters of water gauge, or less than or equal to 5 millimeters of water gauge, or less than or equal to 2.5 millimeters of water gauge, or less than or equal to 2 millimeters of water gauge. Preferably, the RTD of the downstream hollow tubular element is less than or equal to 1 millimeter of water gauge. The RTD of the downstream hollow tubular element may be at least 0 millimeters of water gauge, or at least 0.25 millimeters of water gauge or at least 0.5 millimeters of water gauge or at least 1 millimeter of water gauge.

The flow channel of the downstream hollow tubular element should therefore be free from any components that would obstruct the flow of air in a longitudinal direction. Preferably, the flow channel is substantially empty and particularly preferably the flow channel is empty.

Preferably, the length of the downstream hollow tubular element is at least 3 millimeters, more preferably at least 4 millimeters, more preferably at least 5 millimeters, more preferably at least 6 millimeters. The length of the downstream hollow tubular element is preferably less than 20 millimeters, more preferably less than 15 millimeters, more preferably less than 12 millimeters and more preferably less than 10 millimeters.

The lumen or cavity of the downstream hollow tubular element may have any cross sectional shape. The lumen of the downstream hollow tubular element may have a circular cross sectional shape.

The downstream hollow tubular element may comprise a paper-based material. The downstream hollow tubular element may comprise at least one layer of paper. The paper may be very rigid paper. The paper may be crimped paper, such as crimped heat resistant paper or crimped parchment paper. The downstream hollow tubular element may comprise cardboard. The downstream hollow tubular element may be a cardboard tube.

The downstream hollow tubular element may be a paper tube. The downstream hollow tubular element may be a tube formed from spirally wound paper. The downstream hollow tubular element may be formed from a plurality of layers of the paper. The paper may have a basis weight of at least 50 grams per square meter, at least 60 grams per square meter, at least 70 grams per square meter, or at least 90 grams per square meter.

The downstream hollow tubular element may comprise a polymeric material. For example, the downstream hollow tubular element may comprise a polymeric film. The polymeric film may comprise a cellulosic film. The downstream hollow tubular element may comprise low density polyethylene (LDPE) or polyhydroxyalkanoate (PHA) fibres. Preferably, the downstream hollow tubular element comprises cellulose acetate tow. For example, in preferred embodiments, the downstream hollow tubular element comprises a hollow acetate tube.

Where the downstream hollow tubular element comprises cellulose acetate tow, the cellulose acetate tow may have a denier per filament of between 2 and 4 and a total denier of between 25 and 40.

Where the downstream section further comprises an additional downstream hollow tubular element, as described above, the additional downstream hollow tubular element may be formed of the same material as the downstream hollow tubular element, or a different material.

In certain preferred embodiments, the downstream section may comprise a ventilation zone at a location on the downstream hollow tubular element. In one example, this ventilation zone at a location on the downstream hollow tubular element may be provided instead of a ventilation zone at a location on the hollow tubular cooling element. In another example, the ventilation zone at a location on the downstream hollow tubular element may be provided in addition to the ventilation zone provided at a location on the hollow tubular cooling element.

The ventilation zone at a location along the downstream hollow tubular element may comprise a plurality of perforations through the peripheral wall of the downstream hollow tubular element. Preferably, the ventilation zone at a location along the downstream hollow tubular element comprises at least one circumferential row of perforations. In some embodiments, the ventilation zone may comprise two circumferential rows of perforations. For example, the perforations may be formed online during manufacturing of the aerosol-generating article. Preferably, each circumferential row of perforations comprises from 8 to 30 perforations.

The aerosol-generating article may comprise one or more hollow tubular elements. The one or more hollow tubular elements may form part of a downstream section of the aerosol-generating article arranged downstream of the aerosol-forming substrate portion. The one or more hollow tubular elements may comprise one or both of a hollow acetate tube (HAT) and a fine hollow acetate tube (FHAT). Such hollow tubes are cylindrical components which may be made from cellulose acetate and which are provided with centrally arranged axial holes. The dimensions of the hollow tubes such as their outer diameter or the diameter of the hole vary and can be designed according to the demands of the respective products.

The HAT may have a length of between 6 millimeters and 10 millimeters, preferably between 7 millimeters and 9 millimeters, more preferably about 8 millimeters. The HAT may be arranged downstream of the aerosol-forming substrate portion, preferably downstream of and directly abutting the aerosol-forming substrate portion. The HAT may serve as on ore more of an airflow-cooling element and an airflow-accelerating element.

The FHAT may be arranged downstream of the HAT, preferably downstream of and directly abutting the HAT. The inner diameter of the FHAT may be larger than the inner diameter of the HAT. For example, the inner diameter of the FHAT may be about twice the size of the inner diameter of the HAT. The FHAT may serve as an airflow-decelerating element.

The aerosol-generating article may comprise a mouth-end filter. The mouth-end filter may be arranged downstream of the aerosol-forming substrate portion. The mouth-end filter may be arranged at a proximal end of the aerosol-generating article. The mouth-end filter may be arranged downstream of and directly abutting the FHAT.

The mouth-end filter may comprise a filter material. The filter material may be a filamentary material, for example cellulose acetate. The dernier per filament may be 12. The dernier of the filter material may be 12Y28.

The length of the mouth-end filter along a longitudinal direction of the aerosol-generating article may be between 10 millimeters and 14 millimeters, preferably between 11 millimeters and 13 millimeters, more preferably about 12 millimeters.

The resistance to draw of the mouth-end filter may be between 1 millimeter of water gauge and 100 millimeters of water gauge, preferably between 2 millimeters of water gauge and 50 millimeters of water gauge, more preferably between 5 millimeters of water gauge and 40 millimeters of water gauge, more preferably between 10 millimeters of water gauge and 30 millimeters of water gauge, more preferably between 16 millimeters of water gauge and 20 millimeters of water gauge, more preferably between 17 millimeters of water gauge and 19 millimeters of water gauge, more preferably is about 18 millimeters of water gauge. The resistance to draw of the mouth-end filter be per millimeter length along a longitudinal direction of the aerosol-generating article may be between 0.1 millimeter of water gauge and 20 millimeters of water gauge, preferably between 0.2 millimeter of water gauge and 10 millimeters of water gauge, more between 0.5 millimeter of water gauge and 5 millimeters of water gauge, more between 1 millimeter of water gauge and 2 millimeters of water gauge, more preferably between 1.3 millimeters of water gauge and 1.7 millimeters of water gauge, more preferably between 1.4 millimeter of water gauge and 1.6 millimeters of water gauge, more preferably about 1.5 millimeters of water gauge.

Aerosol-generating articles according to the present disclosure may comprise an upstream section located upstream of the aerosol-forming substrate portion. The upstream section is preferably located immediately upstream of the aerosol-forming substrate portion. The upstream section preferably extends between the upstream end of the aerosol-generating article and the aerosol-forming substrate portion. The upstream section may comprise one or more upstream elements located upstream of the aerosol-forming substrate portion.

The aerosol-generating articles of the present invention preferably comprise an upstream element located upstream of and adjacent to the aerosol-forming substrate portion. The upstream element advantageously prevents direct physical contact with the upstream end of the aerosol-forming substrate portion Furthermore, the presence of an upstream element helps to prevent any loss of the substrate, which may be advantageous, for example, if the substrate contains particulate plant material.

Where the upstream segment of the aerosol-forming substrate portion comprises shredded tobacco, such as tobacco cut filler, the upstream section or element thereof may additionally help to prevent the loss of loose particles of tobacco from the upstream end of the article. This may be particularly important when the shredded tobacco has a relatively low density, for example.

An upstream element may be a porous plug element. Preferably, an upstream element has a porosity of at least 50 percent in the longitudinal direction of the aerosol-generating article. More preferably, an upstream element has a porosity of between 50 percent and 90 percent in the longitudinal direction. The porosity of an upstream element in the longitudinal direction is defined by the ratio of the cross-sectional area of material forming the upstream element and the internal cross-sectional area of the aerosol-generating article at the position of the upstream element.

An upstream element may be made of a porous material or may comprise a plurality of openings. This may, for example, be achieved through laser perforation. Preferably, the plurality of openings is distributed homogeneously over the cross-section of the upstream element.

The porosity or permeability of an upstream element may advantageously be designed in order to provide an aerosol-generating article with a particular overall resistance to draw (RTD) without substantially impacting the filtration provided by other portions of the article.

An upstream element may be formed from a material that is impermeable to air. In such embodiments, the aerosol-generating article may be configured such that air flows into the aerosol-forming substrate portion through suitable ventilation means provided in a wrapper.

In certain preferred embodiments of the invention, it may be desirable to minimise the RTD of an upstream element. For example, this may be the case for articles that are intended to be inserted the cavity of an aerosol-generating device such that the aerosol- forming substrate is externally heated, as described herein. For such articles, it is desirable to provide the article with as low an RTD as possible, so that the majority of the RTD experience by the consumer is provided by the aerosol-generating device and not the article.

The RTD of an upstream element may be less than 30 millimeters of water gauge, or less than 20 millimeters of water gauge, or less than 10 millimeters of water gauge, or less than 5 millimeters of water gauge, or less than 2 millimeters of water gauge. The RTD of an upstream element may be at least 0.1 millimeters of water gauge, or at least 0.25 millimeters of water gauge or at least 0.5 millimeters of water gauge. Preferably, an upstream element has an RTD of less than 2 millimeters of water gauge per millimeter of length, more preferably less than 1.5 millimeters of water gauge per millimeter of length, more preferably less than 1 millimeter of water gauge per millimeter of length, more preferably less than 0.5 millimeters of water gauge per millimeter of length, more preferably less than 0.3 millimeters of water gauge per millimeter of length, more preferably less than 0.2 millimeters of water gauge per millimeter of length.

Preferably, the combined RTD of the upstream section, or upstream element thereof, and the aerosol-forming substrate portion is less than 15 millimeters of water gauge, more preferably less than 12 millimeters of water gauge, more preferably less than 10 millimeters of water gauge.

In certain preferred embodiments, an upstream element is formed of a solid cylindrical plug element having a filled cross-section. Such a plug element may be referred to as a ‘plain’ element. The solid plug element may be porous, as described above, but does not have a tubular form and therefore does not provide a longitudinal flow channel. The solid plug element preferably has a substantially uniform transverse cross section.

In other preferred embodiments, an upstream element is formed of a hollow tubular segment defining a longitudinal cavity providing an unrestricted flow channel. In such embodiments, an upstream element can provide protection for the aerosol-forming substrate, as described above, whilst having a minimal effect on the overall resistance to draw (RTD) and filtration properties of the article.

Preferably, the diameter of the longitudinal cavity of the hollow tubular segment forming an upstream element is at least 3 millimeters, more preferably at least 3.5 millimeters, more preferably at least 4 millimeters and more preferably at least 4.5 millimeters. Preferably, the diameter of the longitudinal cavity is maximised in order to minimise the RTD of the upstream section, or upstream element thereof.

Preferably, the wall thickness of the hollow tubular segment is less than 2 millimeters, more preferably less than 1.5 millimeters and more preferably less than 1 millimeter.

An upstream element of the upstream section may be made of any material suitable for use in an aerosol-generating article. The upstream element may, for example, be made of a same material as used for one of the other components of the aerosol-generating article, such as the downstream filter segment or the hollow tubular cooling element. Suitable materials for forming the upstream element include filter materials, ceramic, polymer material, cellulose acetate, cardboard, zeolite or aerosol-forming substrate. The upstream element may comprise a plug of cellulose acetate. The upstream element may comprise a hollow acetate tube, or a cardboard tube.

Preferably, an upstream element is formed of a heat resistant material. For example, preferably an upstream element is formed of a material that resists temperatures of up to 350 degrees Celsius. This ensures that an upstream element is not adversely affected by the heating means for heating the aerosol-forming substrate.

Preferably, the upstream section, or an upstream element thereof, has an external diameter that is approximately equal to the external diameter of the aerosol-generating article. Preferably, the external diameter of the upstream section, or an upstream element thereof, is between 5 millimeters and 8 millimeters, more preferably between 5.25 millimeters and 7.5 millimeters, more preferably between 5.5 millimeters and 7 millimeters.

Preferably, the upstream section or an upstream element has a length of between 2 millimeters and 10 millimeters, more preferably between 3 millimeters and 8 millimeters, more preferably between 2 millimeters and 6 millimeters. In a particularly preferred embodiment, the upstream section or an upstream element has a length of 5 millimeters.

The upstream section is preferably circumscribed by a wrapper, such as a plug wrap. The wrapper circumscribing the upstream section is preferably a stiff plug wrap, for example, a plug wrap having a basis weight of at least 80 grams per square meter, or at least 100 grams per square meter, or at least 110 grams per square meter. This provides structural rigidity to the upstream section.

The upstream section is preferably connected to the aerosol-forming substrate portion and optionally at least a part of the downstream section by means of an outer wrapper.

The aerosol-generating article may comprise an upstream section comprising a front plug. The front plug may be arranged upstream of and directly abutting the aerosol-forming substrate portion. The front plug may be arranged at a distal end of the aerosol-generating article. The front plug may comprise a filter material. The length of the front plug along a longitudinal direction of the aerosol-generating article may be between 1 millimeters and 10 millimeters, preferably between 3 millimeters and 7 millimeters, more preferably between 4 millimeters and 6 millimeters, more preferably about 5 millimeters. The front plug may be in the form of a full cylinder.

A ratio of substrate wrapper thickness to front plug diameter may be in the range from 0.007 to 0.03, preferably 0.015 to 0.027, more preferably 0.022 to 0.024.

An outer diameter of the front plug may differ from an outer diameter of the substrate wrapper circumscribing the aerosol-forming substrate portion by less than 5 percent, preferably by less than 3 percent, more preferably by less than 1 percent, optionally, wherein the outer diameter of the front plug is 7.1 millimeters.

The front plug may be circumscribed by a front plug wrapper. A ratio of the thickness of the front plug wrapper to the thickness of the substrate wrapper may be 0.7 or less, more preferably 0.5 or less, more preferably 0.3 or less, more preferably 0.2 or less.

It may be that no portion of the front plug is circumscribed by the substrate wrapper.

The resistance to draw of the front plug may be between 1 millimeter of water gauge and 150 millimeters of water gauge, preferably between 1 millimeter of water gauge and 50 millimeters of water gauge, more preferably between 1 millimeter of water gauge and 20 millimeters of water gauge, more preferably between 1 millimeter of water gauge and 10 millimeters of water gauge. The resistance to draw of the front plug may be about 10 millimeters of water gauge or less.

The front plug may assist in maintaining cleanliness of the aerosol-generating device by trapping slurry in the consumable. The front plug may hinder aerosol-forming substrate or a heating element from falling out of the aerosol-generating article.

The aerosol-generating article in accordance with the invention may have an overall length of at least 40 millimeters, or at least 50 millimeters, or at least 60 millimeters.

An overall length of an aerosol-generating article in accordance with the invention may be less than or equal to 90 millimeters, or less than or equal to 85 millimeters, or less than or equal to 80 millimeters.

In some embodiments, an overall length of the aerosol-generating article is preferably from 50 millimeters to 90 millimeters, more preferably from 60 millimeters to 90 millimeters, even more preferably from 70 millimeters to 90 millimeters. In other embodiments, an overall length of the aerosol-generating article is preferably from 50 millimeters to 85 millimeters, more preferably from 60 millimeters to 85 millimeters, even more preferably from 70millimeters to 85 millimeters. In further embodiments, an overall length of the aerosol-generating article is preferably from 50 millimeters to 80 millimeters, more preferably from 60 millimeters to 80 millimeters, even more preferably from 70 millimeters to 80 millimeters. In an exemplary embodiment, an overall length of the aerosol-generating article is 75 millimeters.

In some embodiments, an overall length of the aerosol-generating article is preferably from 40 millimeters to 70 millimeters, more preferably from 45 millimeters to 70 millimeters. In other embodiments, an overall length of the aerosol-generating article is preferably from 40 millimeters to 60 millimeters, more preferably from about 45 millimeters to about 60 millimeters. In further embodiments, an overall length of the aerosol-generating article is preferably from 40 millimeters to 50 millimeters, more preferably from 45 millimeters to 50 millimeters. In an exemplary embodiment, an overall length of the aerosol-generating article is about 45 millimeters.

Preferably, the aerosol-generating article has an external diameter of at least about 5 millimeters. More preferably, the aerosol-generating article has an external diameter of at least 5.25 millimeters. Even more preferably, the aerosol-generating article has an external diameter of at least 5.5 millimeters.

The aerosol-generating article preferably has an external diameter of less than or equal to 8 millimeters. More preferably, the aerosol-generating article has an external diameter of less than or equal to 7.5 millimeters. Even more preferably, the aerosol-generating article has an external diameter of less than or equal to 7 millimeters. The aerosol-generating article may have an external diameter of between 5 millimeters and 8 millimeters, or between 5 millimeters and 7.5 millimeters, or between 5 millimeters and 7 millimeters, or between 5.25 millimeters and 8 millimeters, or between 5.25 millimeters and 7.5 millimeters, or between 5.25 millimeters and 7 millimeters, or between 5.5 millimeters and 8 millimeters, or between 5.5 millimeters and 7.5 millimeters, or between 5.5 millimeters and 7 millimeters.

The external diameter of the aerosol-generating article may be substantially constant over the whole length of the article. As an alternative, different portions of the aerosol-generating article may have different external diameters.

Preferably, the overall RTD of the aerosol-generating article is at least 10 millimeters of water gauge. For example, the overall RTD of the aerosol-generating article may be at least 20 millimeters of water gauge, at least 30 millimeters of water gauge, at least 35 millimeters of water gauge, or at least 40 millimeters of water gauge.

The overall RTD of the aerosol-generating article may be no more than 70 millimeters of water gauge. For example, the overall RTD of the aerosol-generating article may be no more than 65 millimeters of water gauge, no more than 60 millimeters of water gauge, or no more than 55 millimeters of water gauge, or no more than 50 millimeters.

The overall RTD of the aerosol-generating article may be between 10 millimeters of water gauge and 70 millimeters of water gauge. For example, the overall RTD of the aerosol-generating article may be between 20 millimeters of water gauge and 65 millimeters of water gauge, between 30 millimeters of water gauge and 60 millimeters of water gauge, between 35 millimeters of water gauge and 55 millimeters of water gauge, or between 40 millimeters of water gauge and 50 millimeters of water gauge.

In particularly preferred embodiments, one or more of the components of the aerosol-generating article are individually circumscribed by their own wrapper.

In an embodiment, the aerosol-forming substrate portion and the mouthpiece element are individually wrapped. The upstream element, the aerosol-forming substrate portion together with its circumscribing substrate wrapper and the hollow tubular element are then combined together with an outer wrapper. Subsequently, they are combined with the downstream filter element—which has its own wrapper—by means of tipping paper.

Preferably, at least one of the components of the aerosol-generating article is wrapped in a hydrophobic wrapper.

The term “hydrophobic” refers to a surface exhibiting water repelling properties. One useful way to determine this is to measure the water contact angle. The “water contact angle” is the angle, conventionally measured through the liquid, where a liquid/vapour interface meets a solid surface. It quantifies the wettability of a solid surface by a liquid via the Young equation. Hydrophobicity or water contact angle may be determined by utilizing TAPPI T558test method and the result is presented as an interfacial contact angle and reported in “degrees” and can range from near zero to near 180 degrees.

In preferred embodiments, the hydrophobic wrapper is one including a paper layer having a water contact angle of about 30 degrees or greater, and preferably about 35 degrees or greater, or about 40 degrees or greater, or about 45 degrees or greater.

By way of example, the paper layer may comprise PVOH (polyvinyl alcohol) or silicon. The PVOH may be applied to the paper layer as a surface coating, or the paper layer may comprise a surface treatment comprising PVOH or silicon.

The aerosol-generating article may comprise a tipping wrapper at least partly circumscribing the aerosol-forming substrate portion and at least partly circumscribing one or more portions of the aerosol-generating article adjacent to the aerosol-forming substrate portion. The one or more adjacent portions may comprise a front plug.

The tipping wrapper may be a conventional cigarette paper. The tipping wrapper may have a grammage of below 50 grams per square meter. The tipping wrapper may have a thickness of below 70 micrometers or below 50 micrometers. The tipping wrapper may have a thickness of about 65 micrometers and a grammage of about 45 grams per square meter.

The tipping wrapper may be thinner than the substrate wrapper. A ratio of the thickness of the tipping wrapper to the thickness of the substrate wrapper may be 0.7 or less, more preferably 0.5 or less, more preferably 0.3 or less, more preferably 0.2 or less.

The aerosol-generating article may comprise ventilation holes. The ventilation holes may promote nucleation of the aerosol. The ventilation holes may assist in cooling the airflow. The ventilation holes may be provided in the FHAT. The FHAT may comprise 11 ventilation holes each having a diameter of 0.11 millimeter.

The total resistance to draw of the aerosol-generating article may be between 5 millimeters of water gauge and 200 millimeters of water gauge, preferably between 10 millimeters of water gauge and 150 millimeters of water gauge, more preferably between 20 millimeters of water gauge and 100 millimeters of water gauge, more preferably between 80 millimeters of water gauge and 80 millimeters of water gauge, more preferably between 40 millimeters of water gauge and 60 millimeters of water gauge, more preferably between 45 millimeters of water gauge and 55 millimeters of water gauge, more preferably about 48 millimeters of water gauge.

The aerosol-generating article may have a cylindrical shape. The aerosol-forming substrate portion may have a cylindrical shape.

The aerosol-generating article may comprise, in order from a proximal end to a distal end of the article, a mouth-end filter, one or more intermediate elements, an aerosol-forming substrate portion, and, optionally, a front plug. The one or more intermediate elements may comprise one or more of a HAT, a FHAT, and a PLA plug. The total length of the article may be about 45 millimeters and the length of the length of the aerosol-forming substrate portion may be about 11 millimeters. The tipping wrapper may circumscribe the complete article or only a portion thereof.

The aerosol-forming substrate portion may comprise a susceptor. The susceptor may be at least partly circumscribed by the aerosol-forming substrate. The susceptor may be completely surrounded by the aerosol-forming substrate. The susceptor may extend along substantially the entire length of the aerosol-forming substrate portion. This may provide an optimized distribution of heat within the aerosol-forming substrate when the susceptor is heated. The susceptor may comprise a flat planar portion. The susceptor may be a flat planar susceptor strip. The susceptor may comprise a metal or an alloy. The susceptor may comprise aluminum.

As used herein, the term “flat planar” relates to a generally cuboid shape having a height being significantly smaller than a width and a length. For example, the width and length each may be at least twice the height of the cuboid. The height of the flat planar cuboid may also be referred to as the thickness of the susceptor, or of the flat planar portion of the susceptor.

The susceptor element may generally have a thickness from 0.01 millimeter to 2 millimeters, for example from 0.5 millimeter to 2 millimeters. In some embodiments, the susceptor element preferably has a thickness from 10 micrometers to 500 micrometers, more preferably from 10 micrometers to 100 micrometers.

The susceptor may have a thickness from about 35 micrometers to about 85 micrometers. The susceptor may have a thickness from about 45 micrometers to about 75 micrometers. The susceptor may have a thickness from about 55 micrometers to about 65 micrometers.

The susceptor may be an elongate susceptor arranged substantially longitudinally within the aerosol-forming substrate portion.

When used for describing the susceptor, the term “elongate” denotes that the susceptor has a length dimension that is greater than its width dimension or its thickness dimension, for example greater than twice its width dimension or its thickness dimension.

The susceptor may be arranged substantially longitudinally within the aerosol-forming substrate portion. This means that the length dimension of the elongate susceptor is arranged to be approximately parallel to the longitudinal direction of the aerosol-forming substrate, for example within plus or minus 10 degrees of parallel to the longitudinal direction of the aerosol-forming substrate. The elongate susceptor may be positioned in a radially central position within the aerosol-forming substrate portion and extend along the longitudinal axis of the aerosol-forming substrate portion.

The susceptor may be in the form of a pin, rod, strip or blade.

The susceptor may have a length from about 5 millimeters to about 15 millimeters, for example from about 6 millimeters to about 12 millimeters, more preferably from about 8 millimeters to about 10 millimeters. The susceptor may have a length of about 11 millimeters.

The susceptor may have a width of at least about 1 millimeters, more preferably at least about 2 millimeters. Typically, the susceptor may have a width of up to 8 millimeters, preferably of less than or equal to about 6 millimeters.

Preferably, the elongate susceptor element has a length which is the same or shorter than the length of the aerosol-forming substrate portion in which it is incorporated. The length of the susceptor element may be 99% or less, 95% or less, 90% or less, 85% or less, 80% or less 70% or less 60% or less, 50% or less of the length of the aerosol-forming substrate portion in which it is incorporated. The length of the susceptor element may be between 70% and 99%, preferably between 75% and 95%, more preferably between 80% and 95%, more preferably between 85% and 95% of the length of the aerosol-forming substrate portion in which it is incorporated.

When the susceptor has a constant cross-section, for example a circular cross-section, it may have a width or diameter from about 1 millimeter to about 5 millimeters.

When the susceptor has the form of a strip or blade, the strip or blade may have a rectangular cross-section having a width of preferably from about 2 millimeters to about 8 millimeters, more preferably from about 3 millimeters to about 6 millimeters. A susceptor in the form of a strip of blade may have a width of about 4 millimeters.

The elongate susceptor may have a thickness from about 57 micrometers to about 63 micrometers. Even more preferably, the elongate susceptor may have a thickness from about 58 micrometers to about 62 micrometers. Most preferably, the elongate susceptor has a thickness of about 60 micrometers.

The resistance to draw of the aerosol-forming substrate portion may be between 0.1 millimeters of water gauge and 200 millimeters of water gauge, preferably between 1 millimeters of water gauge and 100 millimeters of water gauge, more preferably between 5 millimeters of water gauge and 40 millimeters of water gauge, more preferably between 10 millimeters of water gauge and 30 millimeters of water gauge, more preferably between 17 millimeters of water gauge and 29 millimeters of water gauge, preferably between 20 millimeters of water gauge and 26 millimeters of water gauge, more preferably about 23 millimeters of water gauge. The resistance to draw of the aerosol-forming substrate portion may be 18 millimeters of water gauge or more. The resistance to draw of the aerosol-forming substrate portion may be 23 millimeters of water gauge or more.

Having a low resistance to draw of the aerosol-forming substrate portion, for example less than 10 millimeters of water gauge, may mean that there is only little interaction between the airflow and the aerosol-forming substrate such that there is only little aerosolization. Having a high resistance to draw of the aerosol-forming substrate portion, for example more than 30 millimeters of water gauge, may mean that there is there is a substantial influence of the aerosol-forming substrate portion on the overall resistance to draw of the aerosol-generating article. The resistance to draw of the aerosol-forming substrate portion may vary to some extent from article to article due to manufacturing tolerances. Reducing the influence of the aerosol-forming substrate portion on the overall resistance to draw of the aerosol-generating article may lead to a more constant resistance to draw between different articles.

The resistance to draw of the aerosol-forming substrate portion per millimeter length of the aerosol-forming substrate portion may be between 0.1 millimeter of water gauge and 20 millimeters of water gauge, preferably between 0.2 millimeter of water gauge and 10 millimeters of water gauge, more preferably between 1 millimeter of water gauge and 5 millimeters of water gauge, more preferably between 1.7 millimeters of water gauge and 2.5 millimeters of water gauge, more preferably between 1.9 millimeters of water gauge and 2.3 millimeters of water gauge. The resistance to draw of the aerosol-forming substrate portion per millimeter length along a longitudinal direction of the aerosol-generating article may be about 2.1 millimeters of water gauge.

A total length of the aerosol-forming substrate portion may be between 1 millimeter and 30 millimeters, preferably between 5 millimeter and 16 millimeters, more preferably between 9 millimeters and 13 millimeters, more preferably between 10 millimeters and 12 millimeters, in a direction along the longitudinal axis of the aerosol-generating article. A total length of the aerosol-forming substrate portion may be 11 millimeters or less in a direction along the longitudinal axis of the aerosol-generating article.

A total length of aerosol-generating article may be between 10 millimeters and 150 millimeters, preferably, between 20 millimeters and 1000 millimeters, more preferably between 30 millimeters and 80 millimeters, more preferably between 40 millimeters and 50 millimeters, more preferably between 43 millimeters and 47 millimeters, more preferably about 45 millimeters. A length of the aerosol-forming substrate along a longitudinal direction of the article may be between 22% and 26% of the total length of the aerosol-generating article, preferably about 24% of the total length of the aerosol-generating article.

A ratio between the total length of the aerosol-forming substrate portion and an overall length of the aerosol-generating article may be at least 0.20. Preferably, a ratio between the total length of the aerosol-forming substrate portion and an overall length of the aerosol-generating article is at least 0.25. More preferably, a ratio between the total length of the aerosol-forming substrate portion and an overall length of the aerosol-generating article is at least 0.30.

A ratio between the total length of the aerosol-forming substrate portion and an overall length of the aerosol-generating article is preferably less than or equal to 0.60. Preferably, a ratio between the total length of the aerosol-forming substrate portion and an overall length of the aerosol-generating article is less than or equal to 0.55. More preferably, a ratio between the total length of the aerosol-forming substrate portion and an overall length of the aerosol-generating article is less than or equal to 0.50.

In some embodiments, a ratio between the total length of the aerosol-forming substrate portion and an overall length of the aerosol-generating article is from 0.20 to 0.60, preferably from 0.20 to 0.55, more preferably from 0.20 to 0.50. In other embodiments, a ratio between the total length of the aerosol-forming substrate portion and an overall length of the aerosol-generating article is from 0.25 to 0.60, preferably from 0.25 to 0.55, more preferably from 0.25 to 0.50. In further embodiments, a ratio between the total length of the aerosol-forming substrate portion and an overall length of the aerosol-generating article is from 0.30 to 0.60, preferably from 0.30 to 0.55, more preferably from 0.30 to 0.50.

As used herein, the terms “external diameter” and “outer diameter” of the aerosol-generating article, or a component thereof, may be calculated as the average of a plurality of measurements of the diameter of the aerosol-generating article, or the component thereof, taken at different locations along the length of the aerosol-generating article, or the component thereof.

Preferably, the aerosol-generating article has an external diameter of at least about 5 millimeters. More preferably, the aerosol-generating article has an external diameter of at least 5.25 millimeters. Even more preferably, the aerosol-generating article has an external diameter of at least 5.5 millimeters.

The aerosol-generating article preferably has an external diameter of less than or equal to 8 millimeters. More preferably, the aerosol-generating article has an external diameter of less than or equal to 7.5 millimeters. Even more preferably, the aerosol-generating article has an external diameter of less than or equal to 7 millimeters.

Preferably, the aerosol-generating article has a substantially circular cross-section. Preferably, the aerosol-generating article has a substantially uniform cross-section along the entire length of the aerosol-generating article.

The total density of the aerosol-forming substrate portion may be more than 0.71 milligrams per cubic millimeter. The total density of the aerosol-forming substrate portion may be 0.715 milligrams per cubic millimeter or more. The total density of the aerosol-forming substrate portion may be 0.720 milligrams per cubic millimeter or more. The total density of the aerosol-forming substrate portion may be about 0.725 milligrams per cubic millimeter.

For example, the substrate wrapper may circumscribe a cylindrical volume of 6.77 millimeters in diameter and 11 millimeters in length, i.e. a volume of 396 cubic millimeters. The volume may be filled with aerosol-forming substrate and a susceptor. The total mass of aerosol-forming substrate within the aerosol-forming substrate portion may be 266 milligrams and the total mass of susceptor material within the aerosol-forming substrate portion may be 21.2 milligrams. The total density of the aerosol-forming substrate portion then corresponds to 287.2 milligrams divided by 396 cubic millimeters, i.e. 0.725 milligrams per cubic millimeter.

The total density of the aerosol-generating article at the longitudinal position of the aerosol-forming substrate portion, taking into account also the mass and volume of the substrate wrapper and one or more optional further wrappers circumscribing the substrate wrapper, may be about 0.66 milligrams per cubic millimeter.

A ratio of the total density of the aerosol-forming substrate portion divided by the total density of the of the aerosol-generating article at the longitudinal position of the aerosol-forming substrate portion may be greater than 1.0, preferably greater than 1.05, more preferably 1.09 or greater.

At least 70 percent by volume, preferably at least 75 percent by volume, more preferably at least about 79 percent by volume, of the inner volume of the aerosol-forming substrate portion may be filled with aerosol-forming substrate and, optionally, one or more susceptor elements.

Less than 30 percent by volume, preferably less than 25 percent by volume, more preferably about 21 percent by volume or less, of the inner volume of the aerosol-forming substrate portion may be empty.

The total mass of aerosol-forming substrate in the aerosol-forming substrate portion may be less than 300 milligrams, preferably less than 290 milligrams. The total mass of aerosol-forming substrate in the aerosol-forming substrate portion may be about 266 milligrams. The total mass of aerosol-forming substrate in the aerosol-forming substrate portion may be between 10 milligrams and 3000 milligrams, preferably between 50 milligrams and 1000 milligrams, more preferably between 100 milligrams and 500 milligrams, more preferably between 200 milligrams and 400 milligrams, more preferably between 250 milligrams and 350 milligrams, more preferably between 260 milligrams and 270 milligrams, more preferably between 263 milligrams and 269 milligrams.

The aerosol-forming substrate portion may comprise aerosol-forming substrate and a susceptor. The total mass of susceptor material in the aerosol-forming substrate portion may be between 1 milligrams and 100 milligrams, preferably between 5 milligrams and 50 milligrams, more preferably between 10 milligrams and 40 milligrams, more preferably between 15 milligrams and 25 milligrams, more preferably between 20 milligrams and 23 milligrams, more preferably between 20.5 milligrams and 21.7 milligrams.

The aerosol-forming substrate portion may comprise aerosol-forming substrate and a susceptor, and the total mass of susceptor material in the aerosol-forming substrate portion may be between 20.5 milligrams and 21.7 milligrams and the total mass of aerosol-forming substrate in the aerosol-forming substrate portion may be between 263 milligrams and 269 milligrams.

The density of the aerosol-forming substrate may be more than 800 kilograms per cubic meter, preferably more than 825 kilograms per cubic meter, more preferably may be about 842 kilograms per cubic meter.

The aerosol-forming substrate may be provided in form of a sheet. The sheet of aerosol-forming substrate may be gathered upon insertion into the aerosol-forming substrate portion. The density of the sheet of aerosol-forming substrate may be determined by dividing the grammage of the sheet by the thickness of the sheet before gathering the sheet.

The aerosol-forming substrate may be provided in form of a gathered sheet of homogenized tobacco material.

The sheet of homogenized tobacco material may have a grammage of less than 210 grams per square meter, preferably less than 200 grams per square meter, more preferably about 192 grams per square meter.

The sheet of homogenized tobacco material may have a thickness of more than 215 micrometers, preferably more than 220 micrometers, more preferably about 228 micrometers.

The sheet of homogenized tobacco material may be a casted sheet. The homogenized tobacco material may comprise, prior to the casting process, tobacco particles having an average particle size (D95) of more than 50 micrometers, preferably between more than 50 micrometers and less than 100 micrometers, more preferably between 60 micrometers and 80 micrometers, more preferably between 65 micrometers and 75 micrometers, more preferably about 70 micrometers. This tobacco particle size (D95) may result in a roughened surface of the sheet. This may result in an increased surface area of the sheet. An increased surface may improve aerosolization. This may be particularly advantageous when the total mass of aerosol-forming substrate within the aerosol-forming substrate portion is reduced. As used herein, the term “average particle size (D95)” is used to denote the volume-basis median value of the particle size distribution and is the value of the particle diameter at 95% in the cumulative distribution. The particle size of the particles can be analyzed by laser diffraction method.

The aerosol-forming substrate may comprise tobacco material, from about 1 percent to about 5 percent of a binder, and from about 10 percent to about 30 percent of glycerine, in dry weight basis.

The aerosol-forming substrate portion may define a substantially cylindrical shape. The cylindrical shape of the aerosol-forming substrate portion may have a diameter in a range from about 3 millimeters to about 10 millimeters, preferably from about 6 millimeters to about 8 millimeters, more preferably from about 6.5 millimeters to about 7.5 millimeters, more preferably from about 6.6 millimeters to about 7.0 millimeters, more preferably from about 6.7 millimeters to about 6.9 millimeters, more preferably from about 6.75 millimeters to about 6.85 millimeters. The cylindrical shape of the aerosol-forming substrate portion may have a diameter in a range from about 6.8 millimeters to about 7.1 millimeters, or from about 6.8 millimeters to about 7.0 millimeters.

The invention further relates to an aerosol-generating article, comprising an aerosol-forming substrate in an aerosol-forming substrate portion; and a substrate wrapper at least partly circumscribing the aerosol-forming substrate portion, wherein the substrate wrapper does not extend beyond ends of the aerosol-forming substrate portion in a longitudinal direction of the aerosol-generating article, and wherein the substrate wrapper has a thickness of 50 micrometers or more and a basis weight of less than 50 grams per square meter, preferably of more than 28 grams per square meter and less than 50 grams per square meter.

The invention further relates to an aerosol-generating article, comprising an aerosol-forming substrate in an aerosol-forming substrate portion; and a substrate wrapper at least partly circumscribing the aerosol-forming substrate portion, wherein the substrate wrapper comprises one or more layers having the same length in a longitudinal direction of the aerosol-generating article, and wherein the substrate wrapper has a thickness of 50 micrometers or more and a basis weight of less than 50 grams per square meter, preferably of more than 28 grams per square meter and less than 50 grams per square meter.

The invention further relates to an aerosol-generating system comprising the aerosol-generating article as described herein and an aerosol-generating device. The aerosol-generating device may comprise a heating chamber configured for at least partly inserting the aerosol-generating article into the heating chamber. The aerosol-generating device may comprise an internal heating element arranged for being inserted into the aerosol-generating article, when the aerosol-generating article is at least partly inserted into the heating chamber. The aerosol-generating device may comprise an inductor coil. The inductor coil may at least partly circumscribe the heating chamber. The inductor coil may be arranged to coaxially circumscribe the heating chamber. The inductor coil may be arranged to inductively heat a susceptor element. The susceptor element may be part of an internal heating element of the aerosol-generating device. The susceptor element may be part of the aerosol-generating article. The inductor coil may be arranged to inductively heat a susceptor of the aerosol-generating article when the aerosol-generating article is at least partly inserted into the heating chamber.

As used herein, the term “aerosol-forming substrate” refers to a substrate capable of releasing volatile compounds that can form an aerosol. Such volatile compounds may be released by heating the aerosol-forming substrate. The aerosol-forming substrate may be in solid form or may be in liquid form. The aerosol-forming substrate may be solid or liquid or may comprise both solid and liquid components. An aerosol-forming substrate may be part of an aerosol-generating article. The terms ‘aerosol’ and ‘vapor’ are used synonymously.

The aerosol-forming substrate may comprise one or more of: tobacco, nicotine, an aerosol-generating film, a gel composition, and a flavour agent. The aerosol-forming substrate may comprise homogenised tobacco material such as cast leaf, aerosol-generating films and gel compositions.

The aerosol-forming substrate may comprise one or more aerosol formers. The aerosol former may be any suitable known compound or mixture of compounds that, in use, facilitates formation of a dense and stable aerosol. The aerosol former may be facilitating that the aerosol is substantially resistant to thermal degradation at temperatures typically applied during use of the aerosol-generating article. Suitable aerosol formers are for example: polyhydric alcohols such as, for example, triethylene glycol, 1,3-butanediol, propylene glycol and glycerine; esters of polyhydric alcohols such as, for example, glycerol mono-, di- or triacetate; aliphatic esters of mono-, di- or polycarboxylic acids such as, for example, dimethyl dodecanedioate and dimethyl tetradecanedioate; and combinations thereof. Preferably, the one or more aerosol formers comprise one or both of glycerol and propylene glycol. The one or more aerosol formers may consist of one or both of glycerol and propylene glycol. Preferably, the aerosol-forming substrate comprises glycerol. The terms “glycerine” and “glycerol” are used synonymously herein.

The aerosol-forming substrate may comprise less than or equal to 80 percent by weight of aerosol former on a dry weight basis of the aerosol-forming substrate. The aerosol-forming substrate may comprise less than or equal to 60 percent by weight of aerosol former on a dry weight basis of the aerosol-forming substrate. The aerosol-forming substrate may comprise less than or equal to 40 percent by weight of aerosol former on a dry weight basis of the aerosol-forming substrate. The aerosol-forming substrate may comprise less than or equal to 20 percent by weight, or less than or equal to 15 percent by weight of aerosol former on a dry weight basis of the aerosol-forming substrate.

The aerosol-forming substrate may comprise between 5 percent and 80 percent, or between 5 percent and 60 percent, or between 5 percent and 40 percent, or between 5 percent and 20 percent, or between 5 percent and 15 percent, or between 7 percent and 80 percent, or between 7 percent and 60 percent, or between 7 percent and 40 percent, or between 7 percent and 20 percent, or between 7 percent and 15 percent, or between 10 percent and 80 percent, or between 10 percent and 60 percent, or between 10 percent and 40 percent, or between 10 percent and 20 percent, or between 10 percent and 15 percent by weight of aerosol former on a dry weight basis of the aerosol-forming substrate.

The aerosol-forming substrate may comprise tobacco material. The aerosol-forming substrate may comprise shredded tobacco material. For example, the shredded tobacco material may be in the form of cut filler, as described in more detail below. Alternatively, the shredded tobacco material may be in the form of a shredded sheet of homogenised tobacco material. Suitable homogenised tobacco materials for use in the present invention are described below.

Within the context of the present specification, the term “cut filler” is used to describe to a blend of shredded plant material, such as tobacco plant material, including, in particular, one or more of leaf lamina, processed stems and ribs, and homogenised plant material.

The cut filler may also comprise other after-cut, filler tobacco or casing.

Preferably, the cut filler comprises at least 25 percent of plant leaf lamina, more preferably, at least 50 percent of plant leaf lamina, still more preferably at least 75 percent of plant leaf lamina and most preferably at least 90 percent of plant leaf lamina. Preferably, the plant material is one of tobacco, mint, tea and cloves. Most preferably, the plant material is tobacco. However, the invention is equally applicable to other plant material that has the ability to release substances upon the application of heat that can subsequently form an aerosol.

Preferably, the cut filler comprises tobacco plant material comprising lamina of one or more of bright tobacco, dark tobacco, aromatic tobacco and filler tobacco. With reference to the present invention, the term “tobacco” describes any plant member of the genus Nicotiana.

The cut filler suitable to be used with the present invention generally may resemble cut filler used for conventional smoking articles. The cut width of the cut filler preferably may be between 0.3 millimeter and 2.0 millimeter, or between 0.5 millimeter and 1.2 millimeter, or between 0.6 millimeter and 0.9 millimeter.

Preferably, the strands have a length of between about 10 millimeters and about 40 millimeters before the strands are collated to form the aerosol-forming substrate portion.

In preferred embodiments, the weight of the cut filler is between 80 milligrams and 400 milligrams, preferably between 120 milligrams and 250 milligrams, more preferably between 150 milligrams and 200 milligrams. This amount of cut filler typically allows for sufficient material for the formation of an aerosol.

Preferably, the cut filler is soaked with the aerosol former. Soaking the cut filler can be done by spraying or by other suitable application methods. The aerosol former may be applied to the blend during preparation of the cut filler. For example, the aerosol former may be applied to the blend in the direct conditioning casing cylinder (DCCC). Conventional machinery can be used for applying an aerosol former to the cut filler. Suitable aerosol formers may be those described herein. Preferably, the aerosol former in the cut filler comprises one or both of glycerol and propylene glycol. The aerosol former may consist of glycerol or propylene glycol or of a combination of glycerol and propylene glycol.

The aerosol-forming substrate may comprise homogenised plant material, for example a homogenised tobacco material.

As used herein, the term “homogenised plant material” encompasses any plant material formed by the agglomeration of particles of plant. For example, sheets or webs of homogenised plant material for the aerosol-forming substrate of the present invention may be formed by agglomerating particles of plant material obtained by pulverising, grinding or comminuting plant material. The homogenised plant material may be produced by casting, extrusion, paper making processes or other any other suitable processes known in the art. The homogenised plant material can be provided in any suitable form.

In some embodiments, the homogenised plant material may be in the form of one or more sheets. As used herein with reference to the invention, the term “sheet” describes a laminar element having a width and length substantially greater than the thickness thereof.

The homogenised plant material may be in the form of a plurality of pellets or granules.

The homogenised plant material may be in the form of a plurality of strands, strips or shreds. As used herein, the term “strand” describes an elongate element of material having a length that is substantially greater than the width and thickness thereof. The term “strand” should be considered to encompass strips, shreds and any other homogenised plant material having a similar form. The strands of homogenised plant material may be formed from a sheet of homogenised plant material, for example by cutting or shredding, or by other methods, for example, by an extrusion method.

Where the homogenised plant material is in the form of one or more sheets, as described above, the sheets may be produced by a casting process. Alternatively, sheets of homogenised plant material may be produced by a paper-making process.

The one or more sheets as described herein may each individually have a thickness of between 100 micrometers and 600 micrometers, preferably between 150 micrometers and 300 micrometers, and most preferably between 200 micrometers and 250 micrometers. Individual thickness refers to the thickness of the individual sheet, whereas combined thickness refers to the total thickness of all sheets that make up the aerosol-forming substrate.

The one or more sheets as described herein may each individually have a grammage of between 100 grams per square meter and 600 grams per square meter.

The one or more sheets as described herein may each individually have a density of from 0.3 grams per cubic centimeter to 1.3 grams per cubic centimeter, and preferably from 0.7 grams per cubic centimeter to 1.0 gram per cubic centimeter.

The one or more sheets as described herein may have been one or more of crimped, folded, gathered and pleated.

The one or more sheets of homogenised plant material may be cut into strands as referred to above. In such embodiments, the aerosol-forming substrate comprises a plurality of strands of the homogenised plant material. The strands may be used to form a plug. Typically, the width of such strands is about 5 millimeters, or about 4 millimeters, or about 3 millimeters, or about 2 millimeters or less. The length of the strands may be greater than about 5 millimeters, between about 5 millimeters to about 15 millimeters, about 8 millimeters to about 12 millimeters, or about 12 millimeters. Preferably, the strands have substantially the same length as each other.

The homogenised plant material may comprise between 2.5 percent and 95 percent by weight of plant particles, or between 5 percent and 90 percent by weight of plant particles, or between 10 percent and 80 percent by weight of plant particles, or between 15 percent and 70 percent by weight of plant particles, or between 20 percent and 60 percent by weight of plant particles, or between 30 percent and 50 percent by weight of plant particles, on a dry weight basis.

In certain embodiments of the invention, the homogenised plant material is a homogenised tobacco material comprising tobacco particles. Sheets of homogenised tobacco material for use in such embodiments of the invention may have a tobacco content of at least about 40 percent by weight on a dry weight basis, more preferably of at least about 50 percent by weight on a dry weight basis more preferably at least about 70 percent by weight on a dry weight basis and most preferably at least about 90 percent by weight on a dry weight basis.

With reference to the present invention, the term “tobacco particles” describes particles of any plant member of the genus Nicotiana. The term “tobacco particles” encompasses ground or powdered tobacco leaf lamina, ground or powdered tobacco leaf stems, tobacco dust, tobacco fines, and other particulate tobacco by-products formed during the treating, handling and shipping of tobacco. In a preferred embodiment, the tobacco particles are substantially all derived from tobacco leaf lamina. By contrast, isolated nicotine and nicotine salts are compounds derived from tobacco but are not considered tobacco particles for purposes of the invention and are not included in the percentage of particulate plant material.

In preferred embodiments, the aerosol-forming substrate comprises strands of homogenised tobacco material, wherein the weight of the strands of homogenised tobacco material is between 50 milligrams and 2000 milligrams, preferably between 80 milligrams and 400 milligrams, more preferably between 120 milligrams and 250 milligrams, more preferably between 150 milligrams and 200 milligrams. This amount of strands of homogenised tobacco material typically allows for sufficient material for the formation of an aerosol.

The aerosol-forming substrate may be in the form of an aerosol-generating film comprising a cellulosic based film forming agent, nicotine and an aerosol former. The aerosol-generating film may further comprise a cellulose based strengthening agent. The aerosol-generating film may further comprise water, preferably 30 percent by weight of less of water.

As used herein, the term “film” is used to describe a solid laminar element having a thickness that is less than the width or length thereof. The film may be self-supporting. In other words, a film may have cohesion and mechanical properties such that the film, even if obtained by casting a film-forming formulation on a support surface, can be separated from the support surface. Alternatively, the film may be disposed on a support or sandwiched between other materials. This may enhance the mechanical stability of the film.

The aerosol-generating film may comprise one or more aerosol formers as described herein, preferably the aerosol-former comprises glycerine, or is glycerine. The aerosol-generating film may have an aerosol former content of at least 5 percent by weight on a dry weight basis. The aerosol-generating film may have an aerosol former content of at least 15 percent by weight on a dry weight basis. The aerosol-generating film may have an aerosol former content of at least 20 percent by weight on a dry weight basis. The aerosol-generating film may have an aerosol former content of at least 30 percent by weight on a dry weight basis. Preferably, the aerosol-generating film has an aerosol former content of at least 40 percent by weight on a dry weight basis. More preferably, the aerosol-generating film has an aerosol former content of at least 45 percent by weight on a dry weight basis. More preferably, the aerosol-generating film has an aerosol former content of at least 50 percent by weight on a dry weight basis.

Preferably, the aerosol-generating film has an aerosol former content of no more than 80 percent by weight on a dry weight basis. More preferably, aerosol-generating film has an aerosol former content of no more than 75 percent by weight on a dry weight basis. More preferably, the aerosol-generating film has an aerosol former content of no more than 70 percent by weight on a dry weight basis.

In the context of the present invention the term “cellulose based film-forming agent” is used to describe a cellulosic polymer capable, by itself or in the presence of an auxiliary thickening agent, of forming a continuous film.

Preferably, the cellulose based film-forming agent is selected from the group consisting of hydroxypropyl methylcellulose (HPMC), methylcellulose (MC), ethylcellulose (EC), hydroxyethyl methyl cellulose (HEMC), hydroxyethyl cellulose (HEC), hydroxypropyl cellulose (HPC), and combinations thereof.

More preferably, the cellulose based film-forming agent is selected from the group consisting of hydroxypropyl methylcellulose (HPMC), methylcellulose (MC), ethylcellulose (EC), and combinations thereof.

In particularly preferred embodiments, the cellulose based film-forming agent is HPMC.

The aerosol-generating film may have a cellulose based film-forming agent content of between 10 percent and 40 percent by weight, or between 15 percent and 35 percent by weight, or between 20 percent and 30 percent by weight, on a dry weight basis.

Preferably, the aerosol-generating film further comprises a cellulose based strengthening agent. Preferably, the cellulose based strengthening agent is selected from the group consisting of cellulose fibres, microcrystalline cellulose (MCC), cellulose powder, and combinations thereof.

The aerosol-generating film may have a cellulose based strengthening agent content of between 0.5 percent and 40 percent by weight on a dry weight basis, or between 5 percent and 30 percent by weight on a dry weight basis, or between 10 percent and 25 percent by weight on a dry weight basis.

The aerosol-generating film may further comprise a carboxymethyl cellulose, preferably sodium carboxymethyl cellulose.

The aerosol-generating film may have a carboxymethyl cellulose content of between 1 percent and 15 percent by weight, or between 2 percent and 12 percent by weight, or between 4 percent and 10 percent by weight on a dry weight basis.

The aerosol-generating film preferably comprises nicotine.

As used herein with reference to the invention, the term “nicotine” is used to describe nicotine, a nicotine base or a nicotine salt. In embodiments in which the aerosol-generating film comprises a nicotine base or a nicotine salt, the amounts of nicotine recited herein are the amount of free base nicotine or amount of protonated nicotine, respectively.

The aerosol-generating film may comprise natural nicotine or synthetic nicotine.

The aerosol-generating film may comprise one or more monoprotic nicotine salts.

As used herein with reference to the invention, the term “monoprotic nicotine salt” is used to describe a nicotine salt of a monoprotic acid.

Preferably, the aerosol-generating film comprises at least 0.5 percent by weight of nicotine on a dry weight basis. More preferably, the aerosol-generating film comprises at least 1 percent by weight of nicotine on a dry weight basis. Even more preferably, the aerosol-generating film comprises at least 2 percent by weight of nicotine on a dry weight basis. In addition, or as an alternative, the aerosol-generating film preferably comprises less than 10 percent by weight of nicotine on a dry weight basis. More preferably, the aerosol-generating film comprises less than 8 percent by weight of nicotine on a dry weight basis. More preferably, the aerosol-generating film comprises less than 6 percent by weight of nicotine on a dry weight basis.

For example, the aerosol-generating film may comprise between 0.5 percent and 10 percent by weight of nicotine, or between 1 percent and 8 percent by weight of nicotine, or between 2 percent and 6 percent by weight of nicotine, on a dry weight basis.

The aerosol-generating film may be a substantially tobacco-free aerosol-generating film.

In preferred embodiments, the aerosol-generating film comprises an acid. More preferably, the aerosol-generating film comprises one or more organic acids. Even more preferably, the aerosol-generating film comprises one or more carboxylic acids. In particularly preferred embodiments, the acid is lactic acid, benzoic acid, fumaric acid or levulinic acid.

Preferably, the aerosol-generating film comprises between 0.25 percent and 3.5 percent by weight of an acid, or between 0.5 percent and 3 percent by weight of an acid, or between 1 percent and 2.5 percent by weight of an acid, on a dry weight basis.

The aerosol-generating film may have a thickness from about 0.1 millimeter to about 1 millimeter, more preferably from about 0.1 millimeter to about 0.75 millimeter, even more preferably from about 0.1 millimeter to about 0.5 millimeter. In particularly preferred embodiments, a layer of the film-forming composition is formed that has a thickness from about 50 micrometers to 400 micrometers, more preferably from about 100 micrometers to 200 micrometers.

The aerosol-generating film may optionally be provided on a suitable carrier element. The aerosol-forming substrate may comprise a gel composition that includes nicotine, at least one gelling agent and the aerosol former. The gel composition is preferably substantially tobacco free.

The preferred weight ranges for nicotine in the gel composition are the same as those defined above in relation to aerosol-generating films.

The gel composition preferably comprises at least 50 percent by weight of aerosol former, more preferably at least 60 percent by weight, more preferably at least 70 percent by weight of aerosol former, on a dry weight basis. The gel composition may comprise up to 80 percent by weight of aerosol former. The aerosol former in the gel composition is preferably glycerol.

The gel composition preferably includes at least one gelling agent. Preferably, the gel composition includes a total amount of gelling agents in a range from about 0.4 percent by weight to about 10 percent by weight, or from about 0.5 percent by weight to about 8 percent by weight, or from about 1 percent by weight to about 6 percent by weight, or from about 2 percent by weight to about 4 percent by weight, or from about 2 percent by weight to about 3 percent by weight.

The term “gelling agent” refers to a compound that homogeneously, when added to a 50 percent by weight water/50 percent by weight glycerol mixture, in an amount of about 0.3 percent by weight, forms a solid medium or support matrix leading to a gel. Gelling agents include, but are not limited to, hydrogen-bond crosslinking gelling agents, and ionic crosslinking gelling agents.

The term “hydrogen-bond crosslinking gelling agent” refers to a gelling agent that forms non-covalent crosslinking bonds or physical crosslinking bonds via hydrogen bonding.

The hydrogen-bond crosslinking gelling agent may include one or more of a galactomannan, gelatin, agarose, or konjac gum, or agar. The hydrogen-bond crosslinking gelling agent may preferably include agar.

The term “ionic crosslinking gelling agent” refers to a gelling agent that forms non-covalent crosslinking bonds or physical crosslinking bonds via ionic bonding.

The ionic crosslinking gelling agent may include low acyl gellan, pectin, kappa carrageenan, iota carrageenan or alginate. The ionic crosslinking gelling agent may preferably include low acyl gellan.

The gelling agent may include one or more biopolymers. The biopolymers may be formed of polysaccharides.

Biopolymers include, for example, gellan gums (native, low acyl gellan gum, high acyl gellan gums with low acyl gellan gum being preferred), xanthan gum, alginates (alginic acid), agar, guar gum, and the like. The composition may preferably include xanthan gum. The composition may include two biopolymers. The composition may include three biopolymers. The composition may include the two biopolymers in substantially equal weights. The composition may include the three biopolymers in substantially equal weights.

The gel composition may further include a viscosifying agent. The viscosifying agent combined with the hydrogen-bond crosslinking gelling agent and the ionic crosslinking gelling agent appears to surprisingly support the solid medium and maintain the gel composition even when the gel composition comprises a high level of glycerol.

The term “viscosifying agent” refers to a compound that, when added homogeneously into a 25° C., 50 percent by weight water/50 percent by weight glycerol mixture, in an amount of 0.3 percent by weight, increases the viscosity without leading to the formation of a gel, the mixture staying or remaining fluid.

The gel composition preferably includes the viscosifying agent in a range from about 0.2 percent by weight to about 5 percent by weight, or from about 0.5 percent by weight to about 3 percent by weight, or from about 0.5 percent by weight to about 2 percent by weight, or from about 1 percent by weight to about 2 percent by weight.

The viscosifying agent may include one or more of xanthan gum, carboxymethyl-cellulose, microcrystalline cellulose, methyl cellulose, gum Arabic, guar gum, lambda carrageenan, or starch. The viscosifying agent may preferably include xanthan gum.

The gel composition may further include a divalent cation. Preferably the divalent cation includes calcium ions, such as calcium lactate in solution. Divalent cations (such as calcium ions) may assist in the gel formation of compositions that include gelling agents such as the ionic crosslinking gelling agent, for example. The ion effect may assist in the gel formation. The divalent cation may be present in the gel composition in a range from about 0.1 to about 1 percent by weight, or about 0.5 percent by weight.

The gel composition may further include an acid. The acid may comprise a carboxylic acid. The carboxylic acid may include a ketone group. Preferably the carboxylic acid may include a ketone group having less than about 10 carbon atoms, or less than about 6 carbon atoms or less than about 4 carbon atoms, such as levulinic acid or lactic acid. Preferably this carboxylic acid has three carbon atoms (such as lactic acid).

The gel composition preferably comprises some water. The gel composition is more stable when the composition comprises some water.

Preferably the gel composition comprises between about 8 percent by weight to about 32 percent by weight water, or from about 15 percent by weight to about 25 percent by weight water, or from about 18 percent by weight to about 22 percent by weight water, or about 20 percent by weight water.

Preferably, where a gel composition is used, the aerosol-forming substrate comprises a porous medium loaded with the gel composition. Advantages of a porous medium loaded with the gel composition is that the gel composition is retained within the porous medium, and this may aid manufacturing, storage or transport of the gel composition. It may assist in keeping the desired shape of the gel composition, especially during manufacture, transport, or use.

The term “porous” is used herein to refer to a material that provides a plurality of pores or openings that allow the passage of air through the material.

The porous medium may be any suitable porous material able to hold or retain the gel composition. Ideally the porous medium can allow the gel composition to move within it. In specific embodiments the porous medium comprises natural materials, synthetic, or semi-synthetic, or a combination thereof. In specific embodiments the porous medium comprises sheet material, foam, or fibres, for example loose fibres; or a combination thereof. In specific embodiments the porous medium comprises a woven, non-woven, or extruded material, or combinations thereof. Preferably the porous medium comprises, cotton, paper, viscose, PLA, or cellulose acetate, of combinations thereof. Preferably the porous medium comprises a sheet material, for example, cotton or cellulose acetate. In a particularly preferred embodiment, the porous medium comprises a sheet made from cotton fibres.

The porous medium may be crimped or shredded. The porous medium may be in the form of a sheet, thread or tubular element.

The aerosol-forming substrate may comprise nicotine. The nicotine-containing aerosol-forming substrate may be a nicotine salt matrix.

Preferably, the aerosol-forming substrate comprises plant material and an aerosol former. Preferably, the plant material is a plant material comprising an alkaloid, more preferably a plant material comprising nicotine, and more preferably a tobacco-containing material.

Preferably, the aerosol-forming substrate comprises at least 70 percent of plant material, more preferably at least 90 percent of plant material by weight on a dry weight basis. Preferably, the aerosol-forming substrate comprises less than 95 percent of plant material by weight on a dry weight basis, such as from 90 to 95 percent of plant material by weight on a dry weight basis.

Preferably, the aerosol-forming substrate comprises at least 5 percent of aerosol former, more preferably at least 10 percent of aerosol former by weight on a dry weight basis. Preferably, the aerosol-forming substrate comprises less than 30 percent of aerosol former by weight on a dry weight basis, such as from 5 to 30 percent of aerosol former by weight on a dry weight basis.

In some particularly preferred embodiments, the aerosol-forming substrate comprises plant material and an aerosol former, wherein the substrate has an aerosol former content of between 5% and 30% by weight on a dry weight basis. The plant material is preferably a plant material comprising an alkaloid, more preferably a plant material comprising nicotine, and more preferably a tobacco-containing material. Alkaloids are a class of naturally occurring nitrogen-containing organic compounds. Alkaloids are found mostly in plants, but are also found in bacteria, fungi and animals. Examples of alkaloids include, but are not limited to, caffeine, nicotine, theobromine, atropine and tubocurarine. A preferred alkaloid is nicotine, which may be found in tobacco.

An aerosol-forming substrate may comprise nicotine. An aerosol-forming substrate may comprise tobacco, for example may comprise a tobacco-containing material containing volatile tobacco flavour compounds, which are released from the aerosol-forming substrate upon heating. In preferred embodiments an aerosol-forming substrate may comprise homogenised tobacco material, for example cast leaf tobacco. The aerosol-forming substrate may comprise both solid and liquid components. The aerosol-forming substrate may comprise a tobacco-containing material containing volatile tobacco flavour compounds, which are released from the substrate upon heating. The aerosol-forming substrate may comprise a non-tobacco material. The aerosol-forming substrate may further comprise an aerosol former. Examples of suitable aerosol formers are glycerine and propylene glycol.

As used herein, the term “tobacco material” is used to describe any material comprising tobacco, including, but not limited to, tobacco leaf, tobacco rib, tobacco stem, tobacco stalk, tobacco dust, expanded tobacco, reconstituted tobacco material and homogenised tobacco material.

As used herein, the term “homogenised tobacco” denotes a material formed by agglomerating particulate tobacco. Homogenized tobacco may include reconstituted tobacco or cast leaf tobacco, or a mixture of both. The term “reconstituted tobacco” refers to paper-like material that can be made from tobacco by-products, such as tobacco fines, tobacco dusts, tobacco stems, or a mixture of the foregoing. Reconstituted tobacco can be made by extracting the soluble chemicals in the tobacco by-products, processing the leftover tobacco fibers into a sheet, and then reapplying the extracted materials in concentrated form onto the sheet.

The term “cast leaf” is used herein to refer to a sheet product made by a casting process that is based on casting a slurry comprising plant particles (for example, clove particles, or tobacco particles and clove particles in a mixture) and a binder (for example, guar gum) onto a supportive surface, such as a belt conveyor, drying the slurry and removing the dried sheet from the supportive surface. An example of the casting or cast leaf process is described in, for example, US-A-5,724,998 for making cast leaf tobacco. In a cast leaf process, particulate plant materials are mixed with a liquid component, typically water, to form a slurry. Other added components in the slurry may include fibres, a binder and an aerosol former. The particulate plant materials may be agglomerated in the presence of the binder. The slurry is cast onto a supportive surface and dried to form a sheet of homogenised plant material.

As used herein, the term “flavourant” refers to a composition having organoleptic properties, which provide a sensory experience to the user, for example to enhance the flavour of aerosol. A flavourant can be used to deliver a gustatory sensation (taste), an olfactory sensation (smell), or both a gustatory and an olfactory sensation to the user, for example when inhaling the aerosol.

As used herein, the term “aerosol-generating article” refers to an article comprising an aerosol-forming substrate that is capable of releasing volatile compounds that can form an aerosol. An aerosol-generating article may be disposable. An aerosol-generating article comprising an aerosol-forming substrate comprising tobacco may be referred to herein as a tobacco stick.

As used herein, the term “aerosol-generating device” refers to a device that interacts with an aerosol-forming substrate to generate an aerosol. An aerosol-generating device may interact with one or both of an aerosol-generating article comprising an aerosol-forming substrate, and a cartridge comprising an aerosol-forming substrate. In some examples, the aerosol-generating device may heat the aerosol-forming substrate to facilitate release of volatile compounds from the substrate. An electrically operated aerosol-generating device may comprise an atomiser, such as an electric heater, to heat the aerosol-forming substrate to form an aerosol.

As used herein, the term “aerosol-generating system” refers to the combination of an aerosol-generating device with an aerosol-forming substrate. When the aerosol-forming substrate forms part of an aerosol-generating article, the aerosol-generating system refers to the combination of the aerosol-generating device with the aerosol-generating article. In the aerosol-generating system, the aerosol-forming substrate and the aerosol-generating device cooperate to generate an aerosol.

As used herein, the term “tubular element” is used to denote an elongate element defining a lumen or airflow passage along a longitudinal axis thereof. In particular, the term “tubular” is used herein to encompass any tubular element having a substantially cylindrical cross-section and defining at least one airflow passage establishing an uninterrupted fluid communication between an upstream end of the tubular element and a downstream end of the tubular element. However, it will be understood that alternative geometries of the tubular element may be possible.

As used herein, the terms “upstream” and “front”, and “downstream” and “rear”, are used to describe the relative positions of components, or portions of components, of the aerosol generating article in relation to the direction in which airflows through the aerosol generating article during use thereof. Aerosol generating articles according to the invention comprise a proximal end through which, in use, an aerosol exits the article. The proximal end of the aerosol generating article may also be referred to as the mouth end or the downstream end. The mouth end is downstream of the distal end. The distal end of the aerosol generating article may also be referred to as the upstream end. Components, or portions of components, of the aerosol generating article may be described as being upstream or downstream of one another based on their relative positions between the proximal end of the aerosol generating article and the distal end of the aerosol generating article. The front of a component, or portion of a component, of the aerosol generating article is the portion at the end closest to the upstream end of the aerosol generating article. The rear of a component, or portion of a component, of the aerosol generating article is the portion at the end closest to the downstream end of the aerosol generating article.

As used herein, the term “longitudinal” refers to the direction corresponding to the main longitudinal axis of the aerosol-generating article, which extends between the upstream and downstream ends of the aerosol-generating article.

The term “length” denotes the dimension of a component of the aerosol-generating article in the longitudinal direction. For example, it may be used to denote the dimension of the rod or of the elongate tubular elements in the longitudinal direction.

As used herein with reference to the present invention, the term “transverse” is used to describe the direction perpendicular to the longitudinal direction. Unless otherwise stated, references to the “cross-section” of the aerosol-generating article or a component of the aerosol-generating article refer to the transverse cross-section.

As used herein, the term “proximal” refers to a user-end, or mouth-end of the aerosol-generating article, and the term “distal” refers to the end opposite to the proximal end.

Components of aerosol-generating articles according to the present invention may be described as being upstream or downstream of one another based on their relative positions between the proximal end of the aerosol-generating article and the distal end of the aerosol-generating article.

The aerosol-generating article may comprise one or more susceptor elements. The one or more susceptor elements may be comprised within the aerosol-forming substrate portion. For example, one or more elongate susceptor elements may be arranged substantially longitudinally within the aerosol-forming substrate portion and in thermal contact with the aerosol-forming substrate.

As used herein, a “susceptor” or “susceptor element” means an element that heats up when subjected to an alternating magnetic field. This may be the result of eddy currents induced in the susceptor element, hysteresis losses, or both eddy currents and hysteresis losses. During use, the susceptor element is located in thermal contact or close thermal proximity with an aerosol-forming substrate received in the aerosol-generating device or cartridge. In this manner, the aerosol-forming substrate is heated by the susceptor such that an aerosol is formed.

The susceptor element may be formed from any material that can be inductively heated to a temperature sufficient to generate an aerosol from the aerosol-forming substrate. Preferred susceptor elements comprise a metal or carbon.

A preferred susceptor element may comprise or consist of a ferromagnetic material, for example a ferromagnetic alloy, ferritic iron, or a ferromagnetic steel or stainless steel. A suitable susceptor element may be, or comprise, aluminium.

Suitable susceptor elements may comprise a non-metallic core with a metal layer disposed on the non-metallic core, for example metallic tracks formed on a surface of a ceramic core. A susceptor element may have a protective external layer, for example a protective ceramic layer or protective glass layer encapsulating the susceptor element. The susceptor element may comprise a protective coating formed by a glass, a ceramic, or an inert metal, formed over a core of susceptor element material.

The susceptor element may be arranged in thermal contact with the aerosol-forming substrate of the aerosol-forming substrate portion in which the susceptor element is incorporated. Thus, when the susceptor element heats up the aerosol-forming substrate is heated up and an aerosol is formed. Preferably the susceptor element is arranged in direct physical contact with the aerosol-forming substrate, for example within the aerosol-forming substrate.

The aerosol-generating device suitable for use with an aerosol-generating article as described herein may comprise a heating chamber for receiving at least part of the aerosol-generating article and a heater for heating the aerosol-forming substrate portion of the aerosol-generating article when the aerosol-generating article is received within the heating chamber.

The aerosol-generating device has a distal end and a mouth end. The aerosol-generating device may comprise a body or housing. The body or housing of the aerosol-generating device may define a device cavity for removably receiving the aerosol-generating article at the mouth end of the device.

The device cavity may be referred to as the heating chamber of the aerosol-generating device. The device cavity may extend between a distal end and a mouth, or proximal, end. The distal end of the device cavity may be a closed end and the mouth, or proximal, end of the device cavity may be an open end. An aerosol-generating article may be inserted into the device cavity, or heating chamber, via the open end of the device cavity. The device cavity may be cylindrical in shape so as to conform to the same shape of an aerosol-generating article.

The expression “received within” may refer to the fact that a component or element is fully or partially received within another component or element. For example, the expression “aerosol-generating article is received within the device cavity” refers to the aerosol-generating article being fully or partially received within the cavity of the aerosol-generating device. When the aerosol-generating article is received within the device cavity, the aerosol-generating article may abut the distal end of the device cavity. When the aerosol-generating article is received within the device cavity, the aerosol-generating article may be in substantial proximity to the distal end of the device cavity. The distal end of the device cavity may be defined by an end-wall.

The length of the device cavity may be between 15 millimeters and 80 millimeters, or between 20 millimeters and 70 millimeters, or between 25 millimeters and 60 millimeters, or between 25 millimeters and 50 millimeters.

The length of the device cavity (or heating chamber) may be the same as or greater than the length of the aerosol-forming substrate portion. The length of the device cavity may be the same as or greater than the combined length of the upstream section or element and aerosol-forming substrate portion. Preferably, the length of the device cavity is such that at least 75 percent of the length of the aerosol-forming substrate portion is inserted or received within the device cavity, when the aerosol-generating article is received with the aerosol-generating device. More preferably, the length of the device cavity is such that at least 80 percent of the length of the aerosol-forming substrate portion is inserted or received within the device cavity, when the aerosol-generating article is received with the aerosol-generating device. More preferably, the length of the device cavity is such that at least 90 percent of the length of the aerosol-forming substrate portion is inserted or received within the device cavity, when the aerosol-generating article is received with the aerosol-generating device. This maximises the length of the aerosol-forming substrate portion along which the aerosol-forming substrate can be heated during use, thereby optimising the generation of aerosol from the aerosol-forming substrate and reducing tobacco waste.

The length of the device cavity may be such that the downstream section or a portion thereof is configured to protrude from the device cavity, when the aerosol-generating article received within the device cavity. The length of the device cavity may be such that a portion of the downstream section (such as the hollow tubular cooling element or downstream filter segment) is configured to protrude from the device cavity, when the aerosol-generating article received within the device cavity. The length of the device cavity may be such that a portion of the downstream section (such as the hollow tubular cooling element or downstream filter segment) is configured to be received within the device cavity, when the aerosol-generating article received within the device cavity.

At least 25 percent of the length of the downstream section may be inserted or received within the device cavity, when the aerosol-generating article is received within the device. At least 30 percent of the length of the downstream section may be inserted or received within the device cavity, when the aerosol-generating article is received within the device.

A diameter of the device cavity may be between 4 millimeters and 10 millimeters. A diameter of the device cavity may be between 5 millimeters and 9 millimeters. A diameter of the device cavity may be between 6 millimeters and 8 millimeters. A diameter of the device cavity may be between 6 millimeters and 7 millimeters.

A diameter of the device cavity may be substantially the same as or greater than a diameter of the aerosol-generating article. A diameter of the device cavity may be the same as a diameter of the aerosol-generating article in order to establish a tight fit with the aerosol-generating article.

The device cavity may be configured to establish a tight fit with an aerosol-generating article received within the device cavity. Tight fit may refer to a snug fit. The aerosol-generating device may comprise a peripheral wall. Such a peripheral wall may define the device cavity, or heating chamber. The peripheral wall defining the device cavity may be configured to engage with an aerosol-generating article received within the device cavity in a tight fit manner, so that there is substantially no gap or empty space between the peripheral wall defining the device cavity and the aerosol-generating article when received within the device.

Such a tight fit may establish an airtight fit or configuration between the device cavity and an aerosol-generating article received therein.

With such an airtight configuration, there would be substantially no gap or empty space between the peripheral wall defining the device cavity and the aerosol-generating article for air to flow through.

The tight fit with an aerosol-generating article may be established along the entire length of the device cavity or along a portion of the length of the device cavity.

The aerosol-generating device may comprise an air-flow channel extending between a channel inlet and a channel outlet. The air-flow channel may be configured to establish a fluid communication between the interior of the device cavity and the exterior of the aerosol-generating device. The air-flow channel of the aerosol-generating device may be defined within the housing of the aerosol-generating device to enable fluid communication between the interior of the device cavity and the exterior of the aerosol-generating device. When an aerosol-generating article is received within the device cavity, the air-flow channel may be configured to provide air flow into the article in order to deliver generated aerosol to a user drawing from the mouth end of the article.

The air-flow channel of the aerosol-generating device may be defined within, or by, the peripheral wall of the housing of the aerosol-generating device. In other words, the air-flow channel of the aerosol-generating device may be defined within the thickness of the peripheral wall or by the inner surface of the peripheral wall, or a combination of both. The air-flow channel may partially be defined by the inner surface of the peripheral wall and may be partially defined within the thickness of the peripheral wall. The inner surface of the peripheral wall defines a peripheral boundary of the device cavity.

The air-flow channel of the aerosol-generating device may extend from an inlet located at the mouth end, or proximal end, of the aerosol-generating device to an outlet located away from mouth end of the device. The air-flow channel may extend along a direction parallel to the longitudinal axis of the aerosol-generating device.

The heater may be any suitable type of heater. Preferably, in the present invention, the heater is an external heater.

Preferably, the heater is located at or about the periphery of the heating chamber.

Preferably, the heater externally heats the aerosol-forming substrate portion when the aerosol-generating article is received within the aerosol-generating device. Such an external heater may circumscribe the aerosol-generating article when inserted in or received within the aerosol-generating device.

In some embodiments, the heater is arranged to heat the outer surface of the aerosol-forming substrate portion.

In some embodiments, the heater is arranged for insertion into an aerosol-forming substrate when the aerosol-forming substrate is received within the cavity.

The heater may be positioned within the device cavity, or heating chamber.

The heater may comprise at least one heating element. The at least one heating element may be any suitable type of heating element. In some embodiments, the device comprises only one heating element. In some embodiments, the device comprises a plurality of heating elements.

Suitable materials for forming the at least one resistive heating element include but are not limited to: semiconductors such as doped ceramics, electrically ‘conductive’ ceramics (such as, for example, molybdenum disilicide), carbon, graphite, metals, metal alloys and composite materials made of a ceramic material and a metallic material. Such composite materials may comprise doped or undoped ceramics. Examples of suitable doped ceramics include doped silicon carbides. Examples of suitable metals include titanium, zirconium, tantalum and metals from the platinum group. Examples of suitable metal alloys include stainless steel, nickel-, cobalt-, chromium-, aluminium-titanium-zirconium-, hafnium-, niobium-, molybdenum-, tantalum-, tungsten-, tin-, gallium-, manganese-and iron-containing alloys, and super-alloys based on nickel, iron, cobalt, stainless steel, Timetal® and iron-manganese-aluminium based alloys.

In some embodiments, the at least one resistive heating element comprises one or more stamped portions of electrically resistive material, such as stainless steel. Alternatively, the at least one resistive heating element may comprise a heating wire or filament, for example a Ni—Cr (Nickel—Chromium), platinum, tungsten or alloy wire.

In some embodiments, the at least one heating element comprises an electrically insulating substrate, wherein the at least one resistive heating element is provided on the electrically insulating substrate.

The electrically insulating substrate may comprise any suitable material. For example, the electrically insulating substrate may comprise one or more of: paper, glass, ceramic, anodized metal, coated metal, and Polyimide. The ceramic may comprise mica, Alumina (Al2O3) or Zirconia (ZrO2). Preferably, the electrically insulating substrate has a thermal conductivity of less than or equal to about 40 Watts per meter Kelvin, preferably less than or equal to about 20 Watts per meter Kelvin and ideally less than or equal to about 2 Watts per meter Kelvin.

The heater may comprise a heating element comprising a rigid electrically insulating substrate with one or more electrically conductive tracks or wire disposed on its surface. The size and shape of the electrically insulating substrate may allow it to be inserted directly into an aerosol-forming substrate. If the electrically insulating substrate is not sufficiently rigid, the heating element may comprise a further reinforcement means. A current may be passed through the one or more electrically conductive tracks to heat the heating element and the aerosol-forming substrate.

In some embodiments, the heater comprises an inductive heating arrangement. The inductive heating arrangement may comprise an inductor coil and a power supply configured to provide high frequency oscillating current to the inductor coil. As used herein, a high frequency oscillating current means an oscillating current having a frequency of between about 500 kHz and about 30 MHz. The heater may advantageously comprise a DC/AC inverter for converting a DC current supplied by a DC power supply to the alternating current. The inductor coil may be arranged to generate a high frequency oscillating electromagnetic field on receiving a high frequency oscillating current from the power supply. The inductor coil may be arranged to generate a high frequency oscillating electromagnetic field in the device cavity. In some embodiments, the inductor coil may substantially circumscribe the device cavity. The inductor coil may extend at least partially along the length of the device cavity.

The heater may comprise an inductive heating element. The inductive heating element may be a susceptor element. A susceptor element may be arranged such that, when the aerosol-generating article is received in the cavity of the aerosol-generating device, the oscillating electromagnetic field generated by the inductor coil induces a current in the susceptor element, causing the susceptor element to heat up. In these embodiments, the aerosol-generating device is preferably capable of generating a fluctuating electromagnetic field having a magnetic field strength (H-field strength) of between 1 and 5 kilo amperes per meter (kA m), preferably between 2 and 3 kA/m, for example about 2.5 kA/m. The electrically-operated aerosol-generating device is preferably capable of generating a fluctuating electromagnetic field having a frequency of between 1 and 30 MHz, for example between 1 and 10 MHz, for example between 5 and 7 MHz.

In these embodiments, the susceptor element is preferably located in contact with the aerosol-forming substrate. In some embodiments, a susceptor element is located in the aerosol-generating device. In these embodiments, the susceptor element may be located in the cavity. The aerosol-generating device may comprise only one susceptor element. The aerosol-generating device may comprise a plurality of susceptor elements. In some embodiments, the susceptor element is preferably arranged to heat the outer surface of the aerosol-forming substrate.

The susceptor element may comprise any suitable material, as described above in relation to a susceptor element incorporated within the aerosol-forming substrate portion.

In some embodiments the aerosol-generating device may comprise at least one resistive heating element and at least one inductive heating element. In some embodiments the aerosol-generating device may comprise a combination of resistive heating elements and inductive heating elements.

During use, the heater may be controlled to operate within a defined operating temperature range, below a maximum operating temperature. An operating temperature range between about 150 degrees Celsius and about 300 degrees Celsius in the heating chamber (or device cavity) is preferable. The operating temperature range of the heater may be between about 150 degrees Celsius and about 250 degrees Celsius.

The aerosol-generating device may comprise a power supply. The power supply may be a DC power supply. In some embodiments, the power supply is a battery. The power supply may be a nickel-metal hydride battery, a nickel cadmium battery, or a lithium based battery, for example a lithium-cobalt, a lithium-iron-phosphate or a lithium-polymer battery. However, in some embodiments the power supply may be another form of charge storage device, such as a capacitor. The power supply may require recharging and may have a capacity that allows for the storage of enough energy for one or more user operations, for example one or more aerosol-generating experiences.

Below, there is provided a non-exhaustive list of non-limiting examples. Any one or more of the features of these examples may be combined with any one or more features of another example, embodiment, or aspect described herein.
Example E1: An aerosol-generating article, comprising
an aerosol-forming substrate in an aerosol-forming substrate portion; and
a substrate wrapper at least partly circumscribing the aerosol-forming substrate portion,
wherein the substrate wrapper comprises one or more layers having the same length in a longitudinal direction of the aerosol-generating article, and
wherein the substrate wrapper has a thickness of 50 micrometers or more and a density of 800 kilograms per cubic meter or less.
Example E2: The aerosol-generating article according to Example E1, wherein the combination of all of the one or more layers of the substrate wrapper having the same length defines an overall thickness of the substrate wrapper of 50 micrometers or more and an overall density of the substrate wrapper of 800 kilograms per cubic meter or less.
Example E3: The aerosol-generating article according to Example E1 or Example E2, wherein at least one of the one or more layers of the substrate wrapper has an individual thickness of 50 micrometers or more and an individual density of 800 kilograms per cubic meter or less.
Example E4: The aerosol-generating article according to Example E3, wherein each of the one or more layers of the substrate wrapper having the same length has an individual thickness of 50 micrometers or more and an individual density of 800 kilograms per cubic meter or less.
Example E5: The aerosol-generating article according to any of the preceding examples, wherein the substrate wrapper does not extend beyond longitudinal ends of the aerosol-forming substrate portion in the longitudinal direction of the aerosol-generating article.
Example E6: An aerosol-generating article, comprising
an aerosol-forming substrate in an aerosol-forming substrate portion; and
a substrate wrapper at least partly circumscribing the aerosol-forming substrate portion,
wherein the substrate wrapper does not extend beyond ends of the aerosol-forming substrate portion in a longitudinal direction of the aerosol-generating article, and
wherein the substrate wrapper has a thickness of 50 micrometers or more and a density of 800 kilograms per cubic meter or less.
Example E7: The aerosol-generating article according to any of the preceding examples, wherein the substrate wrapper has a thickness of 60 micrometers or more, preferably 70 micrometers or more, more preferably 75 micrometers or more, more preferably 90 micrometers or more, more preferably 120 micrometers or more, more preferably 145 micrometers or more.
Example E8: The aerosol-generating article according to Example E7, wherein the substrate wrapper has a thickness of between 140 micrometers and 160 micrometers.
Example E9: The aerosol-generating article according to any of the preceding examples, wherein a ratio of substrate wrapper thickness to aerosol-forming substrate portion diameter is in a range from about 1:120 to about 1:20, or about 1:100 to about 1:30, or about 1:80 to about 1:35, or about 1:60 to about 1:40.
Example E10: The aerosol-generating article according to any of the preceding examples, wherein the density of the substrate wrapper is 750 kilograms per cubic meter or less, preferably 700 kilograms per cubic meter or less, more preferably 650 kilograms per cubic meter or less, more preferably 600 kilograms per cubic meter or less, more preferably 550 kilograms per cubic meter or less, more preferably 500 kilograms per cubic meter or less, more preferably 450 kilograms per cubic meter or less, more preferably 400 kilograms per cubic meter or less, more preferably 350 kilograms per cubic meter or less, more preferably is about 320 kilograms per cubic meter.
Example E11: The aerosol-generating article according to any of the preceding examples, wherein the substrate wrapper has a basis weight of less than 60 grams per square meter.
Example E12: The aerosol-generating article according to Example E11, wherein the substrate wrapper has a basis weight of more than 28 grams per square meter and less than 50 grams per square meter.
Example E13: The aerosol-generating article according to any of the preceding examples, wherein the substrate wrapper has a thickness of more than 145 micrometers and a density of 400 kilograms per cubic meter or less.
Example E14: The aerosol-generating article according to any of the preceding examples, wherein the substrate wrapper is perforated.
Example E15: The aerosol-generating article according to any of the preceding examples, wherein the substrate wrapper is embossed.
Example E16: The aerosol-generating article according to any of Examples E1 to E13, wherein the substrate wrapper has a uniform thickness that does not differ at any point by more than about 30 micrometers, or more than about 20 micrometers, or more than about 10 micrometers, or more than about 5 micrometers.
Example E17: The aerosol-generating article according to any of the preceding examples, wherein the substrate wrapper exhibits a permeability of the wrapper in a range from 4000 CORESTA units to 4800 CORESTA units, preferably from 4200 CORESTA units to 4600 CORESTA units, more preferably from 4300 CORESTA units to 4500 CORESTA units, wherein the permeability of cigarette paper is determined by utilizing the International Standard test method ISO 2965:2009 and the result is presented as cubic centimeters per minute per square centimeters and referred to as “CORESTA units”.
Example E18: The aerosol-generating article according to any of the preceding examples, wherein the substrate wrapper has a roughness of between about 50 Bekk seconds and about 1000 Bekk seconds, preferably between about 100 Bekk seconds and about 200 Bekk seconds.
Example E19: The aerosol-generating article according to any of the preceding examples, wherein the substrate wrapper extends along the entire length of the aerosol-forming substrate portion in a direction parallel to the longitudinal direction of the aerosol-generating article.
Example E20: The aerosol-generating article according to any of the preceding examples, wherein the substrate wrapper comprises one or more of cardboard, plastics, and metal foil.
Example E21: The aerosol-generating article according to any of the preceding examples, wherein the substrate wrapper comprises a cellulosic material, for example one or more of paper, wood, textile, natural fibers, and artificial fibers.
Example E22: The aerosol-generating article according to any of the preceding examples, wherein the substrate wrapper comprises a paper layer.
Example E23: The aerosol-generating article according to any of the preceding examples, wherein the substrate wrapper comprises a laminate sheet, preferably, wherein the substrate wrapper is made of a laminate sheet, more preferably, wherein the laminate sheet is a laminate of a paper layer with an aluminum layer.
Example E24: The aerosol-generating article according to any of the preceding examples, wherein the substrate wrapper is formed from a single continuous sheet, preferably a single paper sheet.
Example E25: The aerosol-generating article according to any of Examples E1 to E23, wherein the substrate wrapper is a substrate wrapper system formed from a first individual wrapper sheet and a second individual wrapper sheet, wherein the first individual wrapper sheet comprises a first overlapping region formed by overlapping opposing end portions of the first individual wrapper sheet, wherein the second individual wrapper sheet comprises a second overlapping region formed by overlapping opposing end portions of the second individual wrapper sheet, and wherein the first overlapping region is offset from the second overlapping region by at least about 5 percent, preferably at least about 10 percent, more preferably by at least about 15 percent, more preferably about 40 percent to about 60 percent of a circumference of the aerosol-forming substrate portion.
Example E26: The aerosol-generating article according to any of the preceding examples, wherein a total length of the aerosol-forming substrate portion is 11 millimeters or less.
Example E27: The aerosol-generating article according to any of the preceding examples, wherein the aerosol-forming substrate is provided in form of a gathered sheet of homogenized tobacco material.
Example E28: The aerosol-generating article according to Example E27, wherein the sheet of homogenized tobacco material has a grammage of less than 210 grams per square meter, preferably less than 200 grams per square meter, more preferably about 192 grams per square meter.
Example E29: The aerosol-generating article according to Example E27 or Example E28, wherein the sheet of homogenized tobacco material has a thickness of more than 215 micrometers, preferably more than 220 micrometers, more preferably about 228 micrometers.
Example E30: The aerosol-generating article according to any of Examples E27 to Example E29, wherein the sheet of homogenized tobacco material is a casted sheet, and wherein the homogenized tobacco material comprises, prior to the casting process, tobacco particles having an average particle size (D95) of more than 50 micrometers, preferably between more than 50 micrometers and less than 100 micrometers, more preferably between 60 micrometers and 80 micrometers, more preferably between 65 micrometers and 75 micrometers, more preferably about 70 micrometers.
Example E31: The aerosol-generating article according to any of the preceding examples, wherein the aerosol-forming substrate comprises tobacco material, from about 1 percent to about 5 percent of a binder, and from about 10 percent to about 30 percent of glycerine, in dry weight basis.
Example E32: The aerosol-generating article according to any of the preceding examples, wherein the aerosol-forming substrate portion defines a substantially cylindrical shape having a diameter in a range from about 6.8 millimeters to about 7.1 millimeters, or from about 6.8 millimeters to about 7.0 millimeters.
Example E33: The aerosol-generating article according to any of the preceding examples, wherein the aerosol-forming substrate portion comprises a susceptor, preferably wherein the susceptor is at least partly circumscribed by the aerosol-forming substrate.
Example E34: An aerosol-generating system comprising the aerosol-generating article according to any of the preceding examples and an aerosol-generating device comprising a heating chamber configured for at least partly inserting the aerosol-generating article into the heating chamber.

Features described in relation to one embodiment may equally be applied to other embodiments of the invention.

The invention will be further described, by way of example only, with reference to the accompanying drawings in which:

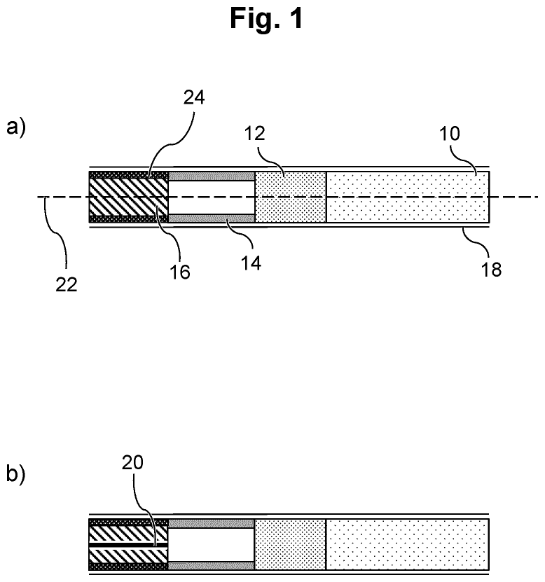

FIGS. 1a and 1b show aerosol-generating articles;

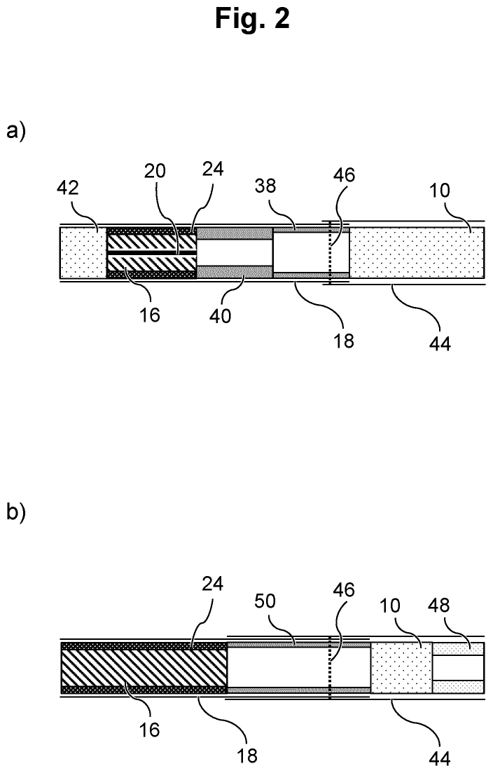

FIGS. 2a and 2b show aerosol-generating articles; and

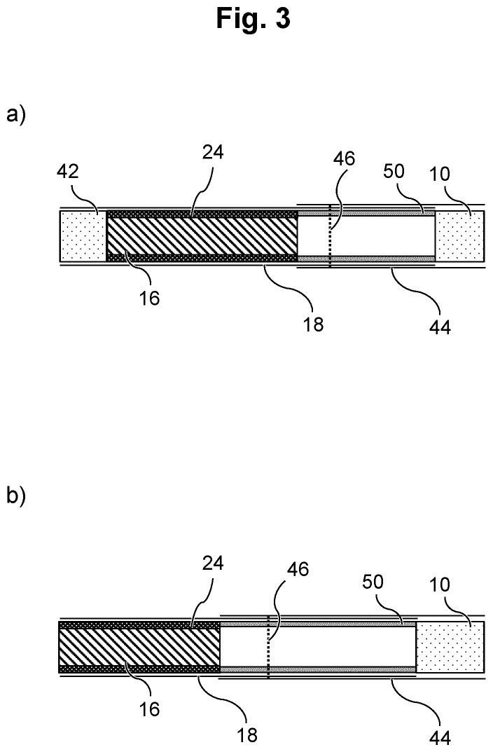

FIGS. 3a and 3b show aerosol-generating articles.

FIG. 1a shows an aerosol-generating article in a cross-sectional view. The aerosol-generating article comprises a mouth-end filter 10 located at a proximal end of the article. The article further comprises a PLA (poly lactic acid) plug 12, a hollow acetate tube 14, and an aerosol-forming substrate portion 16 comprising an aerosol-forming substrate, for example, a gathered sheet of homogenized tobacco. The mouth-end filter 10, PLA plug 12, and the hollow acetate tube 14 form a downstream section downstream of the aerosol-forming substrate portion 16. The article is circumscribed by an outer wrapper 18, for example a tipping wrapper. A center axis 22 extends centrally along a longitudinal direction of the aerosol-generating article. The aerosol-forming substrate portion 16 is circumscribed by a thick and lightweight substrate wrapper 24. The substrate wrapper 24 does not extend beyond longitudinal ends of the aerosol-forming substrate portion 16 in a direction parallel to the center axis 22.

FIG. 1b shows an aerosol-generating article in a cross-sectional view. The article of FIG. 1b is identical to the article of FIG. 1a with the exception that the article of FIG. 1b further comprises a flat planar susceptor 20. The flat planar susceptor 20 is arranged within the aerosol-forming substrate portion 16 and is circumscribed by the aerosol-forming substrate.

FIG. 2a shows an aerosol-generating article in a cross-sectional view. The article of FIG. 2a comprises a mouth-end filter 10, a fine hollow acetate tube 38, a hollow acetate tube 40, an aerosol-forming substrate portion 16 comprising aerosol-forming substrate, and a front plug 42. The mouth-end filter 10, the fine hollow acetate tube 38, and the hollow acetate tube 40 form a downstream section downstream of the aerosol-forming substrate portion 16. The front plug 42 forms an upstream section upstream of the aerosol-forming substrate portion 16.

The aerosol-forming substrate portion 16 may comprise an optional susceptor 20. The front plug 42 may be a filter plug. A distal portion of the article is circumscribed by a tipping wrapper 18 and a proximal portion is circumscribed by a mouthpiece wrapper 44. A circumferential row of ventilation holes 46 is provided in an area where the mouthpiece wrapper 44 overlaps the tipping wrapper 18. The ventilation holes 46 may be provided in one or both of the fine hollow acetate tube 38, the mouthpiece wrapper 44, and the tipping wrapper 18. The aerosol-forming substrate portion 16 is circumscribed by a thick and lightweight substrate wrapper 24.

An outer diameter of the article may be about 7 millimeters, preferably 7.1 millimeters. A total length of the article may be about 45 millimeters. In one embodiment, a length of the mouth-end filter 10 is about 12 millimeters, a length of the fine hollow acetate tube 38 is about 9 millimeters, a length of the hollow acetate tube 40 is about 8 millimeters, a length of the aerosol-forming substrate portion 16 is about 11 millimeters, and a length of the front plug 42 is about 5 millimeters.

FIG. 2b shows an aerosol-generating article in a cross-sectional view. The aerosol-generating article of FIG. 2b may have an overall length of about 75 millimeters and an external diameter of about 6.7 millimeters.

The article of FIG. 2b comprises a hollow mouthpiece tube 48, for example a hollow cylindrical tube made of cellulose acetate, at a proximal end of the article. The hollow mouthpiece tube 48 defines an internal cavity that extends all the way from an upstream end of the hollow mouthpiece tube 48 to a downstream end of a mouth-end filter 10. The internal cavity is substantially empty, and so substantially unrestricted airflow is enabled along the internal cavity. The hollow mouthpiece tube 48 does not substantially contribute to the overall RTD of the aerosol-generating article. The length of the hollow mouthpiece tube 48 may be about 6 millimeters and the external diameter may be about 6.7 millimeters. The wall thickness of the hollow mouthpiece tube 48 may be about 1 millimeter.

The article further comprises a mouth-end filter 10. The mouth-end filter 10 may have a length of about 10 millimeters. An external diameter of the mouth-end filter 10 may be about 6.7 millimeters.

The article further comprises a hollow tube 50, for example a cardboard tube. The hollow tube 50 does not substantially contribute to the overall RTD of the aerosol-generating article. In more detail, the RTD of the hollow tube 50 is about 0 millimeters of water gauge. The hollow tube 50 may have a length of about 25 millimeters or more, an external diameter of about 6.7 millimeters, and an internal diameter of about 6.2 millimeters. Thus, a thickness of a peripheral wall of the hollow tube 50 may be about 0.25 millimeter.

The hollow tube 50 may comprise one or more rows of ventilation holes 46 arranged circumferentially around the hollow tube 50 in a cross-section that is substantially perpendicular to a longitudinal axis of the article. A ventilation level of the aerosol-generating article may be about 75 percent.

At a distal end, the article comprises an aerosol-forming substrate portion 16 comprising an aerosol-forming substrate. The aerosol-forming substrate portion 16 is circumscribed by a thick and lightweight substrate wrapper 24. Additionally, one or more outer wrappers 18, 44 circumscribing at least a portion of the aerosol-generating article may be provided. The one or more outer wrappers 18, 44 may also comprise ventilation holes 46. If present, the outer wrapper 44 may overlie the portion of the outer wrapper 18 that overlies the hollow tube 50. This way, the outer wrapper 44 effectively joins the mouth-end filter 10 to the rest of the components of the article. The width of the outer wrapper 44 may be about 26 millimeters.

In one embodiment, the aerosol-generating article of FIG. 2b has an overall length of about 80 millimeters and an external diameter of about 6.5 millimeters, the hollow tube 50 has a length of about 25 millimeters or more, the mouth-end filter 10 has a length of about 10 millimeters, and the length of the hollow mouthpiece tube 48 is about 6 millimeters.

FIG. 3a shows an aerosol-generating article in a cross-sectional view comprising an aerosol-forming substrate portion 16. The aerosol-forming substrate portion 16 is circumscribed by a thick and lightweight substrate wrapper 24. A downstream section comprises a hollow tube 50 and a mouth-end filter 10. The hollow tube 50 may comprise one or more rows of ventilation holes 46. An upstream section comprises a front plug 42. The front plug 42 may be may be provided in the form of a filled cylindrical plug of cellulose acetate tow, or may be provided in the form of a hollow cylindrical plug of cellulose acetate tow having a wall thickness of about 1 millimeter.

The aerosol-generating article may have an overall length of about 45 millimeters and an external diameter of about 7.2 millimeters. The overall length of the downstream section may be about 20 millimeters to 30 millimeters. The length of the mouth-end filter 10 may be about 7 millimeters. The overall length of the upstream section may be about 5 millimeters.

FIG. 3b shows an aerosol-generating article in a cross-sectional view comprising an aerosol-forming substrate portion 16 at a distal end thereof. The aerosol-forming substrate portion 16 is circumscribed by a thick and lightweight substrate wrapper 24. A downstream section comprises a hollow tube 50 and a mouth-end filter 10. The hollow tube 50 may comprise one or more rows of ventilation holes 46.

The aerosol-generating article of FIG. 3b may have an overall length of about 45 millimeters and an external diameter of about 7.2 millimeters. The hollow tube 50 may have a length of about 20 to 30 millimeters, an external diameter of about 7.2 millimeters, and an internal diameter of about 6.7 millimeters. Thus, a thickness of a peripheral wall of the hollow tube 50 is about 0.25 millimeters. The mouth-end filter 10 may have a length of about 5 millimeters to 7 millimeters and an external diameter of about 7.2 millimeters. The mouth-end filter 10 may comprise a low-density, cellulose acetate filter segment. The RTD of the mouth-end filter 10 may be about 8 millimeters of water gauge. The mouth-end filter 10 may be individually wrapped by a plug wrap (not shown). Additionally, one or more outer wrappers 18, 44 circumscribing at least a portion of the aerosol-generating article may be provided.

1.-15. (canceled)

16. An aerosol-generating article, comprising:
an aerosol-forming substrate in an aerosol-forming substrate portion; and
a substrate wrapper at least partly circumscribing the aerosol-forming substrate portion,
wherein the substrate wrapper comprises one or more layers having a same length in a longitudinal direction of the aerosol-generating article,
wherein the substrate wrapper has a thickness of 50 micrometers or more and a density of 450 kilograms per cubic meter or less, and
wherein the aerosol-forming substrate comprises at least 5 percent of aerosol former by weight on a dry weight basis.

17. The aerosol-generating article according to claim 16, wherein the substrate wrapper has a thickness of 60 micrometers or more.

18. The aerosol-generating article according to claim 16, wherein the substrate wrapper has a thickness of 90 micrometers or more.

19. The aerosol-generating article according to claim 16, wherein the substrate wrapper has a thickness of 145 micrometers or more.

20. The aerosol-generating article according to claim 16, wherein the substrate wrapper has a thickness of between 140 micrometers and 160 micrometers.

21. The aerosol-generating article according to claim 16, wherein a ratio of substrate wrapper thickness to aerosol-forming substrate portion diameter is in a range from about 1:120 to about 1:20, or about 1:100 to about 1:30, or about 1:80 to about 1:35, or about 1:60 to about 1:40.

22. The aerosol-generating article according to claim 16, wherein a density of the substrate wrapper is 400 kilograms per cubic meter or less.

23. The aerosol-generating article according to claim 16, wherein a density of the substrate wrapper is about 320 kilograms per cubic meter.

24. The aerosol-generating article according to claim 16, wherein the substrate wrapper has a basis weight of less than 60 grams per square meter.

25. The aerosol-generating article according to claim 16, wherein the substrate wrapper has a basis weight of more than 28 grams per square meter and less than 50 grams per square meter.

26. The aerosol-generating article according to claim 16, wherein the substrate wrapper has a thickness of more than 145 micrometers and a density of 400 kilograms per cubic meter or less.

27. The aerosol-generating article according to claim 16, wherein the substrate wrapper is perforated.

28. The aerosol-generating article according to claim 16, wherein the substrate wrapper has a uniform thickness that does not differ at any point by more than about 30 micrometers, or more than about 20 micrometers, or more than about 10 micrometers, or more than about 5 micrometers.

29. The aerosol-generating article according to claim 16, wherein the substrate wrapper exhibits a permeability of the wrapper in a range from 4000 CORESTA units to 4800 CORESTA units, and
wherein the permeability of cigarette paper is determined by utilizing the International Standard test method ISO 2965:2009 and the result is presented as cubic centimeters per minute per square centimeters and referred to as “CORESTA units”.

30. The aerosol-generating article according to claim 16,
wherein the substrate wrapper exhibits a permeability of the wrapper in a range from 4300 CORESTA units to 4500 CORESTA units, and
wherein the permeability of cigarette paper is determined by utilizing the International Standard test method ISO 2965:2009 and the result is presented as cubic centimeters per minute per square centimeters and referred to as “CORESTA units”.

31. The aerosol-generating article according to claim 16, wherein the substrate wrapper extends along an entire length of the aerosol-forming substrate portion in a direction parallel to the longitudinal direction of the aerosol-generating article.

32. The aerosol-generating article according to claim 16, wherein the substrate wrapper does not extend beyond longitudinal ends of the aerosol-forming substrate portion in a direction parallel to the longitudinal direction of the aerosol-generating article.

33. The aerosol-generating article according to claim 16, wherein the substrate wrapper is formed from a single continuous sheet.

34. The aerosol-generating article according to claim 16, wherein the substrate wrapper is formed from a single paper sheet.

35. An aerosol-generating system comprising the aerosol-generating article according to claim 16 and an aerosol-generating device comprising a heating chamber configured for at least partly inserting the aerosol-generating article into the heating chamber.
